(19) United States
(12) Patent Application Publication
(10) Pub. No.: US 2016/0000094 A1
(43) Pub. Date: Jan. 7, 2016

(54) BROAD SPECTRUM NATURAL PRESERVATIVE COMPOSITION

(71) Applicant: THE TRUSTEES OF COLUMBIA UNIVERSITY IN THE CITY OF NEW YORK, New York, NY (US)

(72) Inventors: Shanta M. Modak, River Edge, NJ (US); Santoshkumar Dongre, New York, NY (US); Arnab Kumar Ghosh, Jersey City, NJ (US); Lauserpina A. Caraos, Hollis, NY (US)

(73) Assignee: THE TRUSTEES OF COLUMBIA UNIVERSITY IN THE CITY OF NEW YORK, New York, NY (US)

(21) Appl. No.: 14/853,070

(22) Filed: Sep. 14, 2015

Related U.S. Application Data
(63) Continuation-in-part of application No. PCT/US14/29486, filed on Mar. 14, 2014.
(60) Provisional application No. 61/792,386, filed on Mar. 15, 2013.
(60) Provisional application No. 62/073,534, filed on Oct. 31, 2014.

(51) Int. Cl. A01N 65/36; A61K 8/36; A01N 65/08; A61K 8/97; A23L 2/44; A61K 8/34; A61K 8/92; A61Q 19/00; A23L 3/3463; A01N 37/10; A01N 25/02

(52) U.S. Cl. CPC A01N 65/36; A01N 37/10; A61K 8/36; A01N 65/08; A61K 8/97; A01N 25/02; A61K 8/34; A61K 8/922; A61Q 19/00; A23L 3/34635; A23L 2/44; A61K 2800/524; A61K 2800/592; A23V 2002/00

(57) ABSTRACT

Disclosed are combinations of organic acid, botancial extracts, essential oils and alcohol such as benzyl alcohol, phenyl ethanol, ethyl alcohol and combinations thereof, for use in compositions such as, for example, personal care formulations, preservatives for food and beverage products, disinfectants for food, topical disinfectants, surface disinfectants and oral formulations.

### CROSS-REFERENCE TO RELATED APPLICATIONS

This application is a continuation in part of International Application No. PCT/US14/029486, filed Mar. 14, 2014, which claims priority to U.S. Provisional Patent Application No. 61/792,386, filed Mar. 15, 2013; and also claims priority to U.S. Provisional Patent Application No. 62/073,534, filed Oct. 31, 2014, the contents of each of which are hereby incorporated by reference in their entireties, and priority to each of which is claimed.

### 1. INTRODUCTION

Disclosed are combinations of organic acid, one or more botanical extract, one or more essential oil or active constituent thereof, and alcohol such as benzyl alcohol, phenyl ethanol, ethyl alcohol and combinations thereof, for use in compositions such as, for example, personal care formulations, preservatives for food and beverage products, disinfectants for food, topical disinfectants, surface disinfectants and oral formulations. The compositions can further include an odor-masking agent.

### 2. BACKGROUND

In the past, synthetic preservatives such as parabens, imidiazolidinyl urea, DMDM hydantoin, triclosan and quaternary ammonium compounds have been the mainstay of preservatives in cosmetics, toiletries, health and personnel care products. Recently the popularity of traditional preservatives has greatly diminished around the world due to regulatory, environmental and health issues. Concerns over traditional preservatives have resulted in the search for natural alternatives. There has been a significant increase in the use of plant products in many areas of human health such as treating common diseases and as preservatives in personal care products.

The ideal preservative should have broad spectrum antimicrobial activity and the ability to be used in various types of formulations without affecting the function, color or odor of the final product. A preservative composition desirably inactivates bacteria, fungi as well as yeast in both cationic and anionic formulations. Additionally, preservatives should have a low risk of irritation and sensitizing potential. While certain essential oils have antibacterial activity and may be suitable in that regard, most have a distinct odor and are therefore not universally appropriate for use as preservatives in personal care products.

Biosecur® is the first certified organic ingredient for use as a broad-spectrum preservative and disinfectant. Biosecur® is derived from 100% organic citrus fruit, but does not contain grapefruit. It has been reported that, in tests performed according to U.S. Food and Drug Administration's (FDA's) Good Laboratory Practice (GLP) protocol, Biosecur® was found to kill more than 99.999% of common bacteria within 15- to 30-seconds after contact. Furthermore, Biosecur® is reported to provide broad-spectrum antimicrobial efficacy against various bacteria, fungi and yeast.

### 3. SUMMARY

Disclosed herein are preservative compositions comprising effective amounts of one or more organic acid, for example but not limited to, fruit acid such as lactic acid, citric acid, benzoic acid and/or its derivatives, and combinations thereof; one or more botanical extract such as Biosecur®, citrus extract (for example but not limited to, grapefruit seed extract, lemon extract, orange extract and combinations thereof), echinacea extract, portulaca extract, tetrahydrocurcuminoids, wasabi extract, honeysuckle extract, myrrh extract and combinations thereof; one or more essential oil or active constituent thereof, for example but not limited to, cinnamon oil, pine oil, thymol, thyme oil, lemongrass oil, lemon oil, basil oil, oregano oil, rosemary oil, farnesol, cedarwood oil, vetiver, patchouli oil and combinations thereof; and solvent, for example but not limited to, alcohol such as benzyl alcohol, phenyl ethanol, ethyl alcohol and combinations thereof, each of which are preferably obtained from natural sources. These preservatives have been observed to show broad spectrum antimicrobial efficacy.

In certain non-limiting embodiments, the composition comprises between about 0.1-1.0% weight/weight (w/w) citrus extract selected from the group consisting of Biosecur®, grape fruit seed extract, lemon extract, orange extract and combinations thereof; between 0.05-1.0% w/w organic acid, for example but not limited to, fruit acid such as lactic acid, citric acid, benzoic acid and/or its derivatives, and combinations thereat between 0.05-0.1% w/w essential oil or constituents thereof selected from the group consisting of cinnamon oil, pine oil, thymol, thyme oil, lemongrass oil, lemon oil, basil oil, oregano oil, rosemary oil, farnesol, cedarwood oil, vetiver. patchouli oil and combinations thereof; and between 0.05-1.0% w/w solvent selected from the group consisting of phenyl ethanol, ethanol and combinations thereof.

In certain embodiments, the compositions described herein include citrus extract, wherein the citrus extract can include Biosecur®. In certain embodiments, the compositions described herein include citrus extract, wherein the citrus extract does not include Biosecur®.

In certain embodiments, the preservative compositions of the present application comprise (1) between 0-20% w/w or 2-20% w/w of one or more botanical extract selected from curcumin, including the curcumin derivative tetrahydrocurcuminoid and other curcumin compounds, citrus extracts, for example Biosecur® and citrus bioflavonoids, pomegranate extract, golden seal extract, vanilla extract, and vanilla oleo resin; (ii) between 2-20% w/w benzoic acid; (iii) between 1-20% w/w one or more essential oil (or active constituent thereof) selected from thymol, thyme oil, carvacrol and other odorless derivatives of thymol, and cinnamon oil; and (iv) between 5-85% w/w of one or more alcoholic solvent selected from phenyl ethanol, benzyl alcohol, and ethanol. In certain embodiments, the preservative composition comprises one or more agent that is an odor masking agent and/or antioxidant in an amount between 0.01-50% w/w or 1-50% w/w selected from botanical carrier oil, botanical extract, and essential oil. In certain embodiments the odor masking agent/antioxidant is selected from the group consisting of grape seed oil, pomegranate extract, safflower oil, sesame oil, avocado oil, olive oil, jojoba oil, meadowfoam seed oil, almond oil, vanilla oil, vanilla oleo resin, sage oil, lavender oil, peppermint oil, karanja oil, moringa oil, cedar wood oil, vetiver oil, aloe vera extract, honey, portulaca extract, resveratrol, vitamin E (between 0.005-0.01% w/w) and/or vitamin C (between 0.005-0.01% w/w).

In certain non-limiting embodiments, the composition can further comprise between 1-10% w/w of one or more additional essential oil selected from thyme oil, lemongrass oil, and lemon oil. In certain non-limiting embodiments, the composition can further comprise one or more solubilizing agent selected from water and alkanediol (for example, natural 1,3 propanediol (also known as Zemea)).

In certain embodiments, the preservative compositions of the present application comprise (i) low concentrations (between 20-80% w/w) of alcohol selected from ethanol and phenyl ethanol (PE); (ii) between 0-20% w/w or 3-20% w/w of one or more botanical extract selected from tetrahydrocurcuminoid (THC), citrus extract, for example Biosecur® and citrus bioflavonoids, grape seed extract, pomegranate extract, vanilla extract, resveratrol, Echinacea extract, portulaca extract and vanilla oleo resin; (iii) between 5-10% w/w of one or more essential oil (or active constituent thereof) selected from thymol, menthol, cinnamon oil, vanilla oil, and (iv) between 5-20% w/w benzoic acid. In certain embodiments, the compositions can further comprise between 0.5-1% w/w of one or more of calendula extract, resveratrol, Echinacea extract, portulaca extract, and goldenseal extract. In certain embodiments, the compositions can further comprise essential oils selected from vanilla oil, vanilla oleo resin, grape seed oil, and moringa oil. In certain embodiments, the compositions can further comprise one or more odor masking agent in an amount between 0.01-50% w/w or 1-50% w/w selected from botanical carrier oils such as safflower oil, grape seed oil, avocado oil, sesame oil, jojoba oil and combinations thereof, and essential oils (or active constituents thereof) selected from vanilla oil, vanilla oleo resin, lavender oil, pomegranate oil, resveratrol and combinations thereof.

In certain non-limiting embodiments, the preservative compositions described previously are stock solutions, and can be used at a concentration of between 0.1-3.0 or 0.2-2.0% w/w in a personal care product, a topical antimicrobial or antifungal cream for human or veterinary use, a food or beverage preservative or disinfectant, or a pesticide.

### 4. DETAILED DESCRIPTION

Ranges specified herein include their recited limits; for example, a range of between 2 and 4 includes 2 and 4.

For clarity but not by way of limitation, the detailed description is divided into the following subsections,
(i) active agents;
(ii) personal care formulations;
(iii) preservatives for food and beverage products;
(iv) disinfectants for food;
(v) topical disinfectants;
(vi) surface disinfectants; and
(vii) oral formulations
(viii) additional ingredients; and
(ix) methods of use.

### 4.1 Active Agents

Disclosed herein are compositions comprising effective antimicrobial amounts of a combination of one or more botanical extract such as citrus extract selected from Biosecur®, grapefruit seed extract, lemon extract, orange extract and combinations thereof, echinacea extract, portulaca extract, tetrahydrocurcuminoids, wasabi extract, honeysuckle extract, myrrh extract and combinations thereof; organic acid, for example but not limited to, fruit acid such as lactic acid, citric acid, benzoic acid and/or its derivatives, and combinations thereof; one or more essential oil or active constituent thereof and one or more solvent, for example, alcohol such as benzyl alcohol, phenyl ethanol, ethyl alcohol (ethanol) and combinations thereof.

In certain embodiments, a botanical extract, for example, a citrus extract, can be prepared by alcohol/water extraction of a fresh botanical source, for example, a citrus fruit.

In certain embodiments, the essential oils are selected from the group consisting of cinnamon oil (for example cinnamon bark oil and/or cinnamon leaf oil), thyme oil, cedarwood oil, lemon oil, galangal oil, pine oil, basil oil, oregano oil, rosemary oil, cedarwood oil, patchouli oil, vetiver oil, lemongrass oil and combinations thereof. Active constituents of these essential oils include, but are not limited to, cinnamaldehyde, cinnamylacetic ester, cinnamic acid, ethyl cinnamate, methyl chavicol, linalool, beta-caryophyllene, eugenol, thymol, cedrol, cedrene, limonene, d-limonene, geranyl acetate, nerol, geraniol, citral, myrcene, farnesol and combinations thereof.

In certain embodiments, the citrus extract is selected from the group consisting of Biosecur®, lemon extract, orange extract and citrus bioflavonoids.

In the embodiments disclosed in this application, Biosecur® may optionally be substituted with a citrus extract, meaning an extract of juice, or pulp, or rind, or a combination thereof, derived from a citrus fruit or fruits such as orange, lemon and lime, (in certain non-limiting embodiments, not grapefruit and in other non-limiting embodiments, optionally grapefruit) which comprises at least 0.1 percent citric acid and at least 0.2 percent (1 mg/ml) ascorbic acid. In certain embodiments, the citrus extract comprises one or more citrus bioflavonoids.

“Preservative effect” as used herein means “antimicrobial effect.” Antimicrobial effect is static or reduced growth of microbes such as one or more of bacteria (Gram positive and/or Gram negative), protozoa, yeast or fungi. Exemplary microbes which may be inhibited include Staphylococcus aureus, Staphylococcus epidermidis, Enterococcus, Escherichia coli, Pseudomonas aeruginosa, Candida albicans, and Aspergillus niger.

In the formulations disclosed herein, where the weight percentages of ingredients listed do not add up to 1.00 percent, the additional components may include components known in the art for an intended use, for example in personal care products, foods, beverages, and as topical or surface disinfectants. As non-limiting examples, the components may comprise one or more of water or other solvent, thickening agent(s), emollient(s), food product, etc., as the case may be.

In certain non-limiting embodiments, the amount of botanical extract in a composition is between about 0.005 and 5 percent weight/weight (“w/w”), or between about 0.005 and 1 percent w/w, or between about 0.01 and 1 percent w/w, or between about 0.1 and 1.0 percent w/w, or between about 0.05 and 5 percent w/w, or between about 0.05 and 1 percent w/w, or between about 0.1 and 4 percent w/w, or between about 0.15 and 3 percent w/w, or between about 0.2 and 2 percent w/w, or between about 0.3 and 1 percent w/w, or between about 0.5 and 1 percent w/w, or between about 0.2 and 1 percent w/w. In certain embodiments, the botanical extract is selected from the group consisting of Biosecur®, citrus extract, echinacea extract, portulaca extract, tetrahydrocurcuminoids, wasabi extract, honeysuckle extract, myrrh extract and combinations thereof. “About” as used herein means plus or minus 10 percent of the recited value.

In certain embodiments, compositions disclosed herein comprise stock solutions that can be diluted for use comprising one or more botanical extract at a concentration between about 1 and about 90% w/w, or between about 5 and about 80% w/w, or between about 5 and about 70% w/w, or between about 20 and about 70% w/w, or between about 10 and about 50% w/w, or between about 20 and about 40% w/w. In specific non-limiting embodiments the botanical extract is present in the stock at a concentration of between about 40 and about 80% w/w, or between about 5 and about 50% w/w, or between about 5 and about 30% w/w.

In certain non-limiting embodiments, the amount of organic acid in a composition is between about 0.0001 and about 1 percent w/w, or between about 0.0001 and about 0.1 percent w/w, or between about 0.0001 and about 0.2 percent w/w, or between about 0.005 and 3 percent w/w, or between about 0.01 and 2 percent w/w, or between about 0.01 and 0.2 percent w/w, or between about 0.05 and 1 percent w/w, or between about 0.1 and 0.5 percent w/w, or between about 0.2 and 0.5 percent w/w.

In certain embodiments, compositions disclosed herein comprise stock solutions that can be diluted for use comprising organic acid at a concentration between about 1 and about 90% w/w, or between about 5 and about 80 w/w, or between about 20 and about 70% w/w, or between about 10 and about 50% w/w, or between about 20 and about 40% w/w. In certain embodiments, the organic acid is present in the stock at a concentration of between about 0.005 and about 5% w/w, or between about 0.05 and about 2% w/w, or between about 0.01 and about 1% w/w. In specific non-limiting embodiments the organic acid is present in the stock at a concentration of between about 40 and about 80% w/w, or between about 5 and about 30% w/w, or between about 2 and about 30% w/w, or between about 2 and about 20% w/w, or between about 0.01 and about 20% w/w.

In certain non-limiting embodiments, the compositions of the present application comprise an essential oil or active constituent thereof selected from the group consisting of cinnamon oil (for example cinnamon bark oil and/or cinnamon leaf oil), cinnamaldehyde, orange oil, rosemary oil, cedar wood oil, sandal wood oil, patchouli oil, vetiver oil, lemongrass oil, limonene, thymol, thyme oil, ylang ylang oil, lemon oil, fennel oil, fenugreek oil, coriander leaf oil, cedrol, cedrene, citral, pine oil, basil oil, oregano oil, farnesol and combinations thereof. In certain non-limiting embodiments, the amount of essential oil, active constituent thereof, in a composition is between about 0.00005 and about 1 percent w/w, or between about 0.0001 and about 1 percent w/w, or between about 0.0001 and about 0.5 percent w/w, or between about 0.005 and 2 percent w/w, or between about 0.01 and 1 percent w/w, or between about 0.02 and 0.5 percent w/w, or between about 0.03 and 0.1 percent w/w, or between about 0.05 and 0.1 percent w/w.

In certain embodiments, compositions disclosed herein comprise stock solutions that can be diluted for use comprising one or more essential oil and/or active constituent thereof at a concentration between about 1 and about 90% w/w, or between about 5 and about 80% w/w, or between about 20 and about 70% w/w, or between about 10 and about 50% w/w, or between about 20 and about 40% w/w. In certain embodiments, the essential oil and/or active constituent thereof is present in the stock at a concentration of between about 0.005 and about 20% w/w, or between about 0.01 and about 10% w/w, or between about 0.1 and about 5% w/w. In specific non-limiting embodiments the essential oil and/or active constituent thereof is present in the stock at a concentration of between about 0.01 and about 10% w/w, or between about 1 and about 10% w/w.

In certain non-limiting embodiments, the compositions of the present application comprise a solvent, such as an alcohol solvent selected from the group consisting of benzyl alcohol, phenyl ethanol, ethyl alcohol and combinations thereof. In certain non-limiting embodiments, the amount of solvent in a composition is between about 0.001 and 4 percent w/w, or between about 0.005 and 3 percent w/w, or between about 0.01 and 2 percent w/w, or between about 0.01 and 1 percent w/w, or between about 0.05 and 1 percent w/w, or between about 0.1 and 0.5 percent w/w. In certain non-limiting embodiments, the amount of solvent in a composition is between about 0.001 and 10 percent w/w, or between about 0.005 and 8 percent w/w, or between about 0.01 and 5 percent w/w. In specific non-limiting embodiments, the amount of solvent in a composition is between about 0.01 and 6 percent w/w, or between about 0.01 and 1 percent w/w.

In certain embodiments, compositions disclosed herein comprise stock solutions that can be diluted for use comprising a solvent such as an alcohol solvent selected from the group consisting of benzyl alcohol, phenyl ethanol, ethyl alcohol and combinations thereof, at a concentration between about 1 and about 90% w/w, or between about 5 and about 80% w/w, or between about 20 and about 70% w/w, or between about 10 and about 50% w/w, or between about 20 and about 40% w/w. In specific non-limiting embodiments the solvent is present in the stock at a concentration of between about 5 and about 45% w/w, or between about 30 and about 80% w/w, or between about 20 and about 80% w/w, or between about 5 and about 80% w/w.

In certain embodiments, compositions disclosed herein comprise benzoic acid at a concentration between about 0.01 and about 0.2% w/w; Biosecur® at a concentration between about 0.05 and about 1% w/w; thyme oil, thymol, or combinations thereof, at a concentration between about 0.01 and about 1% w/w; and an alcohol solvent selected from the group consisting of benzyl alcohol, phenyl ethanol, ethanol and combinations thereof, at a concentration between about 0.01 and about 1% w/w.

In certain embodiments, compositions disclosed herein comprise stock solutions that can be diluted for use comprising benzoic acid at a concentration between about 2 and about 20% w/w; Biosecur® at a concentration between about 20 and about 70% w/w; thyme oil, thymol, or combinations thereof, at a concentration between about 1 and about 10% w/w; and an alcohol solvent selected from the group consisting of benzyl alcohol, phenyl ethanol, ethanol and combinations thereof, at a concentration between about 5 and about 45% w/w.

In certain embodiments, compositions disclosed herein comprise benzoic acid at a concentration between about 0.0001 and about 0.1% w/w; Biosecur® at a concentration between about 0.005 and about 1% w/w; one or more essential oil or active constituent thereof, selected from the group consisting of cinnamon oil, thyme oil, thymol, cinnamaldehyde and combinations thereof, at a concentration between about 0.0001 and about 0.5% w/w; and an alcohol solvent selected from the group consisting of benzyl alcohol, phenyl ethanol, ethanol and combinations thereof, at a concentration between about 0.01 and about 1% w/w.

In certain embodiments, compositions disclosed herein comprise stock solutions that can be diluted for use comprising benzoic acid at a concentration between about 0.01 and about 1% w/w; Biosecur® at a concentration between about 5 and about 30% w/w; one or more essential oil or active constituent thereof, selected from the group consisting of cinnamon oil, thyme oil, thymol, cinnamaldehyde and combinations thereof, at a concentration between about 0.01 and about 10% w/w; and an alcohol solvent selected from the group consisting of benzyl alcohol, phenyl ethanol, ethanol and combinations thereof, at a concentration between about 20 and about 80% w/w.

In certain embodiments, compositions disclosed herein comprise benzoic acid at a concentration between about 0.01 and about 0.2% w/w; one or more botanical extract selected from the group consisting of portulaca extract, tetrahydrocurcumanoids and combinations thereof, at a concentration between about 0.01 and about 1% w/w; thyme oil, thymol, or combinations thereof, at a concentration between about 0.01 and about 1% w/w; and an alcohol solvent selected from the group consisting of benzyl alcohol, phenyl ethanol, ethanol and combinations thereof, at a concentration between about 0.01 and about 1% w/w.

In certain embodiments, compositions disclosed herein comprise stock solutions that can be diluted for use comprising benzoic acid at a concentration between about 2 and about 20% w/w; one or more botanical extract selected from the group consisting of portulaca extract, tetrahydrocurcumanoids and combinations thereof, at a concentration between about 5 and about 50% w/w; thyme oil, thymol, or combinations thereof, at a concentration between about 1 and about 10% w/w; and an alcohol solvent selected from the group consisting of benzyl alcohol, phenyl ethanol, ethanol and combinations thereof, at a concentration between about 30 and about 80% w/w.

In certain embodiments, compositions disclosed herein comprise benzoic acid at a concentration between about 0.0001 and about 0.2% w/w; one or more botanical extract selected from the group consisting of Biosecur®, citrus extract, portulaca extract, Tetrahydocurcumanoids (THC) and derivatives thereof, echinacea extract, and combinations thereof, in an amount between about 0.005 and 1% w/w; an essential oil, or active constituent thereof, selected from the group consisting cinnamon oil, lemon oil, thyme oil, galangal oil, thymol, cinnamaldehyde and combinations thereof, in an amount between about 0.0001 and 1% w/w; and an alcohol solvent selected from the group consisting of benzyl alcohol, phenyl ethanol, ethanol and combinations thereof, at a concentration between about 0.01 and about 1% w/w.

In certain embodiments, compositions disclosed herein comprise stock solutions that can be diluted for use comprising benzoic acid at a concentration between about 0.01 and about 20% w/w; one or more botanical extract selected from the group consisting of Biosecur®, citrus extract, portulaca extract, Tetrahydocurcumanoids (THC) and derivatives thereof, echinacea extract, and combinations thereof, in an amount between about 5 and 70% w/w; an essential oil, or active constituent thereof, selected from the group consisting cinnamon oil, lemon oil, thyme oil, galangal oil, thymol, cinnamaldehyde and combinations thereof, in an amount between about 0.01 and 10% w/w; and an alcohol solvent selected from the group consisting of benzyl alcohol, phenyl ethanol, ethanol and combinations thereof, at a concentration between about 5 and about 80% w/w.

In certain embodiments, the compositions disclosed herein comprise between about 0.1-1.0% w/w citrus extract selected from the group consisting of Biosecur®, grapefruit seed extract, lemon extract, orange extract and combinations thereof; between 0.05-1.0% w/w organic acid, for example but not limited to, fruit acid such as lactic acid, citric acid, benzoic acid and/or its derivatives, and combinations thereof; between 0.05-0.1% w/w essential oil or constituents thereof selected from the group consisting of cinnamon oil, pine oil, thymol, thyme oil, lemongrass oil, lemon oil, basil oil, oregano oil, rosemary oil, farnesol, cedarwood oil, vetiver. patchouli oil and combinations thereof; and between 0.05-1.0% w/w solvent selected from the group consisting of phenyl ethanol, ethanol and combinations thereof.

Any of the compositions of the present application may further comprise an agent that masks the odor of the composition or otherwise enhances the olfactory appeal of the composition. In certain embodiment the making agent is an essential oil. In certain embodiments, the masking agent is in an amount between about 0.01 and 1, or between about 0.05 and 1 percent weight/weight.

In certain non-limiting embodiments, the composition does not further comprise antimicrobial effective amounts of any of the following antimicrobial agents: quaternary ammonium compounds such as but not limited to benzalkonium chloride, benzethenium chloride, or cetylpyridinium chloride, biguanides such as chlorhexidine (e.g. chlorhexidine free base, chlorhexidine gluconate or chlorhexidine acetate) or polyhexamethylenebiguanide, chlorinated phenols, triclosan, chlorhexidine or chlorhexidine salt, phenoxyethanol, povidone iodine, aliphatic alcohol, or parachlorometaxylenol.

In certain non-limiting embodiments, the composition does further comprise antimicrobial effective amounts of one or more of the following antimicrobial agents: quaternary ammonium compounds such as but not limited to benzalkonium chloride, benzethenium chloride, or cetylpyridinium chloride, biguanides such as chlorhexidine (e.g. chlorhexidine free base, chlorhexidine gluconate or chlorhexidine acetate) or polyhexamethylenebiguanide, chlorinated phenols, triclosan, chlorhexidine or chlorhexidine salt, phenoxyethanol, povidone iodine, aliphatic alcohol, or parachlorometaxylenol.

In certain non-limiting embodiments, the composition does not contain any fruit acid.

In certain embodiments, the compositions described herein include citrus extract, wherein the citrus extract can include Biosecur®. In certain embodiments, the compositions described herein include citrus extract, wherein the citrus extract does not include Biosecur®.

In certain non-limiting embodiments, the composition does not contain any lauroyl arginate, ethyl lauroyl arginate, glycerol monolaurate, glyceryl laurate and/or propylene glycol monolaurate. In certain non-limiting embodiments, the composition comprises lauroyl arginate, ethyl lauroyl arginate, glycerol monolaurate, glyceryl laurate and/or propylene glycol monolaurate at a concentration below 0.000005 weight percent.

In certain non-limiting embodiments, the composition does not comprise benzyl alcohol.

In certain embodiments, the preservative compositions of the present application comprise (i) between 0.05-50, or between 1-30, or between 2-20, or between 2-10 or between 2-5% w/w of one or more botanical extract selected from curcumin, including the curcumin derivative tetrahydrocurcuminoid and other curcumin compounds, citrus extracts, for example Biosecur® and citrus bioflavonoids, pomegranate extract, golden seal extract, vanilla extract, and vanilla oleo resin; (ii) between 0.05-50, or between 1-30, or between 2-20, or between 2-10 or between 2-5% w/w benzoic acid; (iii) between 0.05-50, or between 1-30, or between 2-20 or between 2-10% w/w one or more essential oil (or active constituent thereof) selected from thymol, thyme oil, carvacrol and other odorless derivatives of thymol, and cinnamon oil; and (iv) between 1-90. or between 5-85 or between 10-70 or between 15-60 or between 20-50% w/w of one or more alcoholic solvent selected from phenyl ethanol, benzyl alcohol, and ethanol. In certain embodiments, the preservative composition comprises one or more agent that is an odor masking agent and/or antioxidant in an amount between 0.005-70, or between 0.5-60, or between 1-50, or between 2-20, or between 5-10% w/w selected from botanical carrier oil, botanical extract, and essential oil. In certain embodiments the odor masking agent/antioxidant is selected from the group consisting of grape seed oil, pomegranate extract, safflower oil, sesame oil, avocado oil, olive oil, jojoba oil, meadowfoam seed oil, almond oil, vanilla oil, vanilla oleo resin, sage oil, lavender oil, peppermint oil, karanja oil, moringa oil, cedar wood oil, vetiver oil, aloe vera extract, honey, portulaca extract, resveratrol, vitamin E (for example, between 0.005-0.01% w/w) and/or vitamin C (for example, between 0.005-0.01% w/w).

In certain non-limiting embodiments, the composition can further comprise between 1-10% w/w of one or more additional essential oil selected from thyme oil, lemongrass oil, and lemon oil. In certain non-limiting embodiments, the composition can further comprise one or more solubilizing agent selected from water and alkanediol (for example, natural 1,3 propanediol (also known as Zemea)).

In certain embodiments, the preservative compositions of the present application comprise (i) low concentrations (or, for example, between 1-90. or between 5-85 or between 10-70 or between 15-60 or between 20-50, or between 20-80% w/w) of alcohol selected from ethanol and phenyl ethanol (PE); (ii) between 0.05-50, or between 1-30, or between 2-20, or between 2-10, or between 3-10 or between 2-5% w/w of one or more botanical extract selected from tetrahydrocurcuminoid (THC), citrus extract, for example Biosecur® and citrus bioflavonoids, grape seed extract, pomegranate extract, vanilla extract, resveratrol, Echinacea extract, portulaca extract and vanilla oleo resin; (iii) between 0.05-50, or between 1-30, or between 2-20, or between 2-10, or between 5-10% w/w of one or more essential oil (or active constituent thereof) selected from thymol, menthol, cinnamon oil, vanilla oil, and; and (iv) between 0.05-50, or between 1-30, or between 2-20, or between 5-20, or between 2-10 or between 2-5% w/w benzoic acid. In certain embodiments, the compositions can further comprise between 0.5-1% w/w of one or more of calendula extract, resveratrol, Echinacea extract, portulaca extract, and goldenseal extract. In certain embodiments, the compositions can further comprise essential oils selected from vanilla oil, vanilla oleo resin, grape seed oil, and moringa oil. In certain embodiments, the compositions can further comprise one or more odor masking agent at a concentration of between 0.005-70, or between 0.5-60, or between 1-50, or between 2-20, or between 5-10% w/w selected from botanical carrier oils such as safflower oil, grape seed oil, avocado oil, sesame oil, and jojoba oil; and essential oils selected from vanilla oil, vanilla oleo resin, lavender oil, pomegranate oil, and resveratrol.

In certain non-limiting embodiments, the preservative compositions described previously are stock solutions, and can be used at a concentration of between 0.1-3.0 or 0.2-2.0% w/w in a personal care product, a topical antimicrobial or antifungal cream for human or veterinary use, a food or beverage preservative or disinfectant, or a pesticide.

As a non-limiting example, the stock preservative can be incorporated into the final product at a use level of between about 0.7 and 1.2% weight/weight (w/w) (such that the preservative constitutes between about 0.7 and 1.2% (w/w) of the final product), or at a use level of between about 0.1 and 0.5% w/w, or at a use level of between about 0.1 and 0.5% w/w, or at a use level of between about 0.7 and 1% w/w, or at a use level of between about 1.0 and 2.0% w/w, or at a use level of about 1.5% w/w. In certain embodiments, the stock preservative can be incorporated into the final product at a use level of about 0.2% w/w. Throughout this document, “% w/w stock” refers to the percent amount, weight to weight, in a stock solution, and “% w/w final” refers to the percent amount, weight to weight, in the final product.

In one set of non-limiting embodiments, the composition comprises the following components:

| Ingredients | % w/w |
|---|---|
| Benzyl alcohol | 0.05-1.0 |
| Cinnamon Bark oil | 0.03-0.1 |
| Portulaca extract | 0.2-2.0 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one specific non-limiting embodiment, the composition comprises the following components:

| Ingredients | % w/w |
|---|---|
| Benzyl alcohol | 0.5 |
| Cinnamon Bark oil | 0.05 |
| Portulaca extract | 0.5 |
| Ethanol | 0.2 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Cinnamon Bark oil | 0.03-0.1 |
| Portulaca extract | 0.2-2.0 |
| Benzoic acid | 0.1-0.5 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one specific non-limiting embodiment, the composition comprises the following components:

| Ingredients | % w/w |
|---|---|
| Benzyl alcohol | 0.5 |
| Cinnamon Bark oil | 0.05 |
| Portulaca extract | 0.5 |
| Benzoic acid | 0.1 |
| Ethanol | 0.2 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Thymol | 0.03-0.1 |
| Portulaca extract | 0.2-2.0 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one specific non-limiting embodiment, the composition comprises the following components:

| Ingredients | % w/w |
|---|---|
| Benzyl alcohol | 0.5 |
| Thymol | 0.05 |
| Portulaca extract | 0.5 |
| Ethanol | 0.2 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Thymol | 0.03-0.1 |
| Portulaca extract | 0.2-2.0 |
| Benzoic acid | 0.1-0.5 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one specific non-limiting embodiment, the composition comprises the following components:

| Ingredients | % w/w |
|---|---|
| Benzyl alcohol | 0.5 |
| Thymol | 0.05 |
| Portulaca extract | 0.5 |
| Benzoic acid | 0.1 |
| Ethanol | 0.2 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Lemongrass oil | 0.03-0.3 |
| Portulaca extract | 0.2-2.0 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one specific non-limiting embodiment, the composition comprises the following components:

| Ingredients | % w/w |
|---|---|
| Benzyl alcohol | 0.5 |
| Lemongrass oil | 0.05 |
| Portulaca extract | 0.5 |
| Ethanol | 0.2 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Lemongrass oil | 0.03-0.1 |
| Portulaca extract | 0.2-2.0 |
| Benzoic acid | 0.1-0.5 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one specific non-limiting embodiment, the composition comprises the following components:

| Ingredients | % w/w |
|---|---|
| Benzyl alcohol | 0.5 |
| Lemongrass oil | 0.05 |
| Portulaca extract | 0.5 |
| Benzoic acid | 0.1 |
| Ethanol | 0.2 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Thymol | 0.03-0.1 |
| Honey suckle extract | 0.2-2.0 |
| Benzoic acid | 0.1-0.5 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Benzyl alcohol | 0.05-1.0 |
| Thymol | 0.03-0.1 |
| Wasabi extract | 0.2-2.0 |
| Benzoic acid | 0.1-0.5 |
| Ethanol | 0.05-1.0 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w |
|---|---|
| Thymol | 0.05 |
| Portulaca extract | 0.5 |
| Benzoic acid | 0.1 |
| Ethanol | 0.3 |

In certain embodiments, the composition further comprises an odor masking agent. In certain embodiments, the odor masking agent is selected from the group consisting of patchouli oil, vetiver oil, sandal wood oil and sandal wood oil related fragrances.

In one set of non-limiting embodiments, the composition comprises the following components:

| Ingredients | % w/w stock |
|---|---|
| Solvent | 5-45 |
| Benzoic acid | 2-20 |
| Citrus extract | 20-70 |
| Essential oil | 1-10 |

In one set of non-limiting embodiments, the composition comprises the following components:

| Ingredients | % w/w stock |
|---|---|
| Solvent | 18.75 |
| Benzoic acid | 12.5 |
| Citrus extract | 62.5 |
| Essential oil | 6.25 |

In one set of non-limiting embodiments, the composition comprises the following components:

| Ingredients | % w/w stock |
|---|---|
| Solvent | 5-45 |
| Benzoic acid | 2-20 |
| Botanical extract | 20-70 |
| Essential oil | 1-10 |

In one set of non-limiting embodiments, the composition comprises the following components:

| Ingredients | % w/w stock |
|---|---|
| Solvent | 23.52 |
| Benzoic acid | 11.76 |
| Botanical extract | 58.82 |
| Essential oil | 5.88 |

In one set of non-limiting embodiments, the composition comprises the following components:

| Ingredients | % w/w stock |
|---|---|
| Phenyl ethanol | 31.57 |
| Biosecur ® | 52.63 |
| Benzoic acid | 10.52 |
| Thymol | 5.26 |

In one set of non-limiting embodiments, the composition comprises the following components:

| Ingredients | % w/w final |
|---|---|
| Phenyl ethanol | 0.3 |
| Biosecur ® | 0.5 |
| Benzoic acid | 0.1 |
| Thymol | 0.05 |

In one set of non-limiting embodiments, the composition comprises the following components:

| Ingredients | % w/w stock |
|---|---|
| Biosecur ® | 66.66 |
| Benzoic acid | 13.33 |
| Thymol | 6.66 |
| Solvent | 13.33 |

In one set of non-limiting embodiments, the composition comprises the following components:

| Ingredients | % w/w final |
|---|---|
| Biosecur ® | 0.5 |
| Benzoic acid | 0.1 |
| Thymol | 0.05 |
| Solvent | 0.1 |

In one set of non-limiting embodiments, the composition comprises the following components:

| Ingredients | % w/w stock |
|---|---|
| Biosecur ® | 70 |
| Benzoic acid | 10 |
| Thymol | 5 |
| Solvent | 15 |

In one set of non-limiting embodiments, the composition comprises the following components:

| Ingredients | % w/w final |
|---|---|
| Biosecur ® | 0.7 |
| Benzoic acid | 0.1 |
| Thymol | 0.05 |
| Solvent | 0.15 |

In one set of non-limiting embodiments, the composition comprises the following components:

| Ingredients | % w/w stock |
|---|---|
| Phenyl ethanol | 31.57 |
| Benzoic acid | 10.52 |
| Thymol | 5.26 |
| Lemon extract | 52.63 |

In one set of non-limiting embodiments, the composition comprises the following components:

| Ingredients | % w/w final |
|---|---|
| Phenyl ethanol | 0.3 |
| Benzoic acid | 0.1 |
| Thymol | 0.05 |
| Lemon extract | 0.5 |

In one set of non-limiting embodiments, the composition comprises the following components:

| Ingredients | % w/w stock |
|---|---|
| Solvent | 13.33 |
| Portulaca extract | 66.66 |
| Benzoic acid | 13.33 |
| Cinnamon oil | 6.66 |

In one set of non-limiting embodiments, the composition comprises the following components:

| Ingredients | % w/w final |
|---|---|
| Solvent | 0.1 |
| Portulaca extract | 0.5 |
| Benzoic acid | 0.1 |
| Cinnamon | 0.05 |

In one set of non-limiting embodiments, the composition comprises the following components:

| Ingredients | % w/w stock |
|---|---|
| Phenyl ethanol | 31.57 |
| Portulaca extract | 52.63 |
| Benzoic acid | 10.52 |
| Cinnamon oil | 5.26 |

In one set of non-limiting embodiments, the composition comprises the following components:

| Ingredients | % w/w final |
|---|---|
| Phenyl ethanol | 0.3 |
| Portulaca extract | 0.5 |
| Benzoic acid | 0.1 |
| Cinnamon oil | 0.05 |

In one set of non-limiting embodiments, the composition comprises the following components:

| Ingredients | % w/w stock |
|---|---|
| Phenyl ethanol | 42.85 |
| Echinacea extract | 21.42 |
| Benzoic acid | 14.28 |
| Thymol | 7.14 |
| Alcohol | 14.28 |

In one set of non-limiting embodiments, the composition comprises the following components:

| Ingredients | % w/w final |
|---|---|
| Phenyl ethanol | 0.3 |
| Echinacea extract | 0.15 |
| Benzoic acid | 0.1 |
| Thymol | 0.05 |
| Alcohol | 0.1 |

In one set of non-limiting embodiments, the composition comprises the following components:

| Ingredients | % w/w stock |
|---|---|
| Solvent | 30.0-80.0 |
| Benzoic acid | 2-20 |
| Botanical extract | 5-50 |
| Essential oil | 1-10 |

In one set of non-limiting embodiments, the composition comprises the following components:

| Ingredients | % w/w final |
|---|---|
| Solvent | 71.52 |
| Benzoic acid | 14.20 |
| Botanical extract | 7.14 |
| Essential oil | 7.14 |

In one set of non-limiting embodiments, the composition comprises the following components:

| Ingredients | % w/w stock |
|---|---|
| Phenyl ethanol | 71.52 |
| Tetrahydrocurcumanoids | 7.14 |
| Benzoic acid | 14.20 |
| Thymol | 7.14 |

In one set of non-limiting embodiments, the composition comprises the following components:

| Ingredients | % w/w final |
|---|---|
| Phenyl ethanol | 0.5 |
| Tetrahydrocurcumanoids | 0.05 |
| Benzoic acid | 0.1 |
| Thymol | 0.05 |

In one set of non-limiting embodiments, the composition comprises the following components:

| Ingredients | % w/w stock |
|---|---|
| Phenyl ethanol | 42.81 |
| Tetrahydrocurcumanoids | 7.14 |
| Benzoic acid | 14.20 |
| Portulaca extract | 28.57 |
| Thymol | 7.14 |

In one set of non-limiting embodiments, the composition comprises the following components:

| Ingredients | % w/w final |
|---|---|
| Phenyl ethanol | 0.3 |
| Tetrahydrocurcumanoids | 0.05 |
| Benzoic acid | 0.1 |
| Portulaca extract | 0.2 |
| Thymol | 0.05 |

In one set of non-limiting embodiments, the composition comprises the following components:

| Ingredients | % w/w stock |
|---|---|
| Phenyl ethanol | 57.14 |
| Tetrahydrocurcumanoids | 7.14 |
| Benzoic acid | 14.20 |
| Portulaca extract | 14.28 |
| Thymol | 7.14 |

In one set of non-limiting embodiments, the composition comprises the following components:

| Ingredients | % w/w final |
|---|---|
| Phenyl ethanol | 0.4 |
| Tetrahydrocurcumanoids | 0.05 |
| Benzoic acid | 0.1 |
| Portulaca extract | 0.1 |
| Thymol | 0.05 |

### 4.2 Personal Care Formulations

Disclosed herein are compositions that may be comprised in personal care products to impart antimicrobial activity and accordingly act as preservatives.

In certain embodiments the composition is a stock solution comprising the recited active agents and a suitable solvent. Non-limiting examples of suitable solvents include water, ethanol, glycerin, octoxyglycerin, and mixtures thereof.

Provided herein is a method for producing a preservative or antimicrobial effect in a personal care product comprising incorporating a preservative formulation as set forth herein into the product.

Personal care products comprising the above ingredients include, but are not limited to, creams such as but not limited to hand cream, lotions and splashes, hair care products, makeup (for example but not limited to foundation, powder, lipstick and eye makeup such as shadow, liner and mascara) and other cosmetic products, gels, tonics, shampoos, soaps (e.g., liquid or bar soap), and wipe formulations. The formulations may be applied to humans or non-human animals (for example, for veterinary or agricultural purposes, for example, as a veterinary cream, veterinary splash, etc.).

In certain non-limiting embodiments, the preservative compositions of the present application are incorporated into a base comprising the following components:

| Ingredient | % weight/weight (w/w) |
|---|---|
| Water | 75.5 |
| UcareJr 30 M | 0.24 |
| Incroquat TMS | 2.4 |
| Polawax NF | 2.4 |
| Petrolatum | 4.8 |
| Stearyl alcohol | 6.6 |
| Polyoxyl 40 stearate | 1.6 |
| Propylene glycol | 1.6 |
| Isopropyl myristate | 3.2 |
| Sorbitan oleate | 1.6 |
| pH 6.5 | |

In certain non-limiting embodiments, the preservative compositions of the present application are incorporated into a base comprising the following components:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH 5.0 | |

In certain non-limiting embodiments, the composition is a soap comprising the following components:

| Ingredients | % w/w (range) |
|---|---|
| Hydroxypropyl Methyl Cellulose (Methocel | 0.0-0.3 |
| 40-202 PCG) | |
| Poly (ethylene oxide) [Polyox WSR N-750] | 0.0-0.3 |
| Polyquaternium-10 (U-care polymer JR-30M) | 0.1-0.3 |
| Pluracare F87 | 0.1-0.2 |
| Cocamido propyl betaine | 5.0-7.0 |
| Glycerin | 1.0-3.0 |
| Propylene glycol | 1.0-3.0 |
| Incromine oxide | 1.0-3.0 |
| SDA-40B/Alcohol 3C | 5.0-20.0 |
| Zemea | 0.0-10.0 |
| Grape fruit seed extract | 0.0-1.0 |
| Curcumin | 0.0-0.5 |
| Lemon oil | 0.05-0.5 |
| Lemon grass oil | 0.0-0.3 |
| Orange oil | 0.0-0.2 |
| Thymol | 0.05-0.2 |
| Menthol | 0.0-0.1 |
| Benzoic acid | 0.0-0.2 |
| Lactic acid/Citric acid | 0.0-1.0 |
| Farnesol | 0.0-0.5 |
| Aloe vera gel (1X) | 1.0-5.0 |
| Volume adjusted to 100% with water | |

In certain specific non-limiting embodiments, the composition is a soap comprising the following specific components:

### Specific Formulation—1

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Menthol | 0.04 |
| Benzoic acid | 0.1 |
| Aloe vera gel (1X) | 2.0 |
| Volume adjusted to 100% with water | |

### Specific Formulation—2

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 3.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Menthol | 0.04 |
| Lactic acid | 0.5 |
| Aloe vera gel (1X) | 2.0 |
| Volume adjusted to 100% with water | |

### Specific Formulation—3

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Menthol | 0.04 |
| Lactic acid | 0.5 |
| Farnesol | 0.3 |
| Aloe vera gel (1X) | 2.0 |
| Volume adjusted to 100% with water | |

### Specific Formulation—4

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Menthol | 0.04 |
| Benzoic acid | 0.005 |
| Lactic acid | 0.5 |
| Aloe vera gel (1X) | 2.0 |
| Volume adjusted to 100% with water | |

### Specific Formulation—5

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Benzoic acid | 0.005 |
| Lactic acid | 0.5 |
| Aloe vera gel (1X) | 2.0 |
| Volume adjusted to 100% with water | |

### Specific Formulation—6

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Lactic acid | 0.5 |
| Aloe vera gel (1X) | 2.0 |
| Volume adjusted to 100% with water | |

### Specific Formulation—7

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.2 |
| Thymol | 0.1 |
| Lactic acid | 0.5 |
| Farnesol | 0.3 |
| Aloe vera gel (1X) | 2.0 |
| Volume adjusted to 100% with water | |

### Specific Formulation—8

| Ingredients | % w/w |
|---|---|
| Hydroxypropyl Methyl Cellulose (Methocel | 0.1 |
| 40-202 PCG) | |
| Poly (ethylene oxide) [Polyox WSR N-750] | 0.1 |
| Polyquaternium-10 (U-care polymer JR-30M) | 0.2 |
| Pluracare F87 | 0.1 |
| Cocamido propyl betaine | 5.0 |
| Glycerin | 1.0 |
| Incromine oxide | 2.0 |
| SDA-40B | 10.0 |
| Zemea | 4.0 |
| Grape fruit seed extract | 0.5 |
| Curcumin | 0.05 |
| Lemon oil | 0.1 |
| Lemongrass oil | 0.3 |
| Orange oil | 0.1 |
| Thymol | 0.1 |
| Lactic acid | 0.5 |
| Farnesol | 0.3 |
| Aloe vera gel (1X) | 2.0 |
| Volume adjusted to 100% with water | |

### Specific Formulation—9

| Ingredients | % w/w |
|---|---|
| Glycerin | 1.0 |
| Olive oil | 7.5 |
| Coconut oil | 7.5 |
| Shea butter | 7.5 |
| Sodium hydroxide | 4.0 |
| SDA-40B | 5.0 |
| Zemea | 4.0 |
| Grape fruit seed extract | 0.5 |
| Curcumin | 0.05 |
| Lemon oil | 0.1 |
| Lemongrass oil | 0.3 |
| Orange oil | 0.1 |
| Thymol | 0.1 |
| Lactic acid | 0.5 |
| Farnesol | 0.3 |
| Aloe vera gel (1X) | 2.0 |
| Volume adjusted to 100% with water | |

In certain non-limiting embodiments, the composition is a cosmetic preservative comprising the following components:

### Cosmetic Preservative—General Composition P

| Ingredients | % w/w (range) |
|---|---|
| Alcohol (phenyl ethanol, benzyl alcohol, and/or | 20-80 |
| ethanol) | |
| Organic acid | 7-20 |
| Essential oil (or active constituent thereof) | 3-20 |
| Botanical derivative/extract | 2.5-20 |
| Odor masking carrier botanical oils | 3-50 |
| Odor masking essential oil | 0.01-0.10 |

In certain embodiments, the use level of the formulation in a product is 1.0-2.0% w/w.

In certain non-limiting embodiments, the composition is a cosmetic preservative comprising the following components:

### Cosmetic Preservative P-V

| Ingredients | % w/w |
|---|---|
| Ethanol | 59.31 |
| Phenyl ethanol | 18.75 |
| Benzoic acid | 12.5 |
| Teytrahydrocurcumin | 3.125 |
| Thymol | 6.25 |
| Vanilla oleo resin | 0.0625 |

In certain embodiments, the use level of the formulation in a product is 1.0-2.0% w/w.

In certain non-limiting embodiments, the composition is a cosmetic preservative comprising the following components:

### Cosmetic Preservative P-GRS

| Ingredients | % w/w |
|---|---|
| Ethanol | 53.125 |
| Phenyl ethanol | 18.75 |
| Benzoic acid | 12.5 |
| Teytrahydrocurcumin | 3.125 |
| Thymol | 6.25 |
| Grape seed oil | 6.25 |

In certain embodiments, the use level of the formulation in a product is 1.0-2.0% w/w.

In certain non-limiting embodiments, the composition is a cosmetic preservative comprising the following components:

### Cosmetic Preservative P-PS

| Ingredients | % w/w |
|---|---|
| Ethanol | 20 |
| Phenyl ethanol | 56.19 |
| Benzoic acid | 12.5 |
| Tetrahydrocurcumin | 3.125 |
| Thymol | 6.25 |
| Safflower oil | 3.125 |
| Pomegranate oil | 0.0625 |

In certain embodiments, the use level of the formulation in a product is 1.0-2.0% w/w.

In certain non-limiting embodiments, the composition is a cosmetic preservative comprising the following components:

### Cosmetic Preservative P-E

| Ingredients | % w/w |
|---|---|
| Ethanol | 58.6 |
| Phenyl ethanol | 18.75 |
| Benzoic acid | 12.5 |
| Teytrahydrocurcumin | 3.125 |
| Thymol | 6.25 |
| Echinacea | 0.78 |

In certain embodiments, the use level of the formulation in a product is 1.0-2.0% w/w.

In certain non-limiting embodiments, the composition is a cosmetic preservative comprising the following components:

### Cosmetic Preservative P-E Gol

| Ingredients | % w/w |
|---|---|
| Ethanol | 59.31 |
| Phenyl ethanol | 18.75 |
| Benzoic acid | 12.5 |
| Tetrahydrocurcumin | 3.125 |
| Thymol | 6.25 |
| Golden seal | 0.0625 |

In certain embodiments, the use level of the formulation in a product is 1.0-2.0% w/w.

In certain non-limiting embodiments, the composition is a cosmetic preservative comprising the following components:

### Cosmetic Preservative P-PS

| Ingredients | % w/w |
|---|---|
| Ethanol | 20 |
| Phenyl ethanol | 56.19 |
| Benzoic acid | 12.5 |
| Tetrahydrocurcumin | 3.125 |
| Thymol | 6.25 |
| Safflower oil | 3.125 |
| Pomegranate oil | 0.0625 |

In certain embodiments, the use level of the formulation in a product is 1.0-2.0% w/w.

In certain non-limiting embodiments, the composition is a cosmetic preservative comprising the following components:

### Cosmetic Preservative P-B1

| Ingredients | % w/w |
|---|---|
| Biosecur A 520 D | 15.0 |
| Benzyl alcohol | 30.0 |
| Thymol | 5.0 |
| Benzoic acid | 10 |
| Alcohol | 41.25 |

In certain embodiments, the use level of the formulation in a product is 1.0-2.0% w/w.

In certain non-limiting embodiments, the composition is a cosmetic preservative comprising the following components:

### Cosmetic Preservative P-BS

| Ingredients | % w/w |
|---|---|
| Biosecur A 520 D | 15.0 |
| Benzyl alcohol | 30.0 |
| Thymol | 5.0 |
| Benzoic acid | 10 |
| Alcohol | 40.0 |
| Sage oil | 1.25 |

In certain embodiments, the use level of the formulation in a product is 1.0-2.0% w/w.

In certain non-limiting embodiments, the composition is a cosmetic preservative comprising the following components:

### Cosmetic Preservative PB-V1

| Ingredients | % w/w |
|---|---|
| Biosecur A 520 D | 15.0 |
| Benzyl alcohol | 30.0 |
| Thymol | 5.0 |
| Benzoic acid | 10 |
| Alcohol | 40.0 |
| Vanilla oleo resin | 1.25 |

In certain embodiments, the use level of the formulation in a product is 1.0-2.0% w/w.

In certain non-limiting embodiments, the composition is a cosmetic preservative comprising the following components:

### Cosmetic Preservative 2

| Ingredients | % w/w in stock |
|---|---|
| Phenyl ethanol | 18.1 |
| Thymol | 6.0 |
| Tetrahydrocurcumanoids | 3.0 |
| Benzoic acid | 12.0 |
| Ethanol | 54.8 |
| Safflower oil | 6.0 |
| Vanilla oil | 0.125 |

In certain non-limiting embodiments, the composition is a cosmetic preservative comprising the following components:

### Cosmetic Preservative 4

| Ingredients | % w/w in stock |
|---|---|
| Phenyl ethanol | 18.1 |
| Thymol | 6.0 |
| Tetrahydrocurcumanoids | 3.0 |
| Benzoic acid | 12.0 |
| Ethanol | 54.8 |
| Safflower oil | 6.0 |
| Vanilla oleoresin | 0.125 |
| Pomegranate extract | 0.062 |

In certain non-limiting embodiments, the composition is a cosmetic preservative comprising the following components:

### Cosmetic Preservative 6

| Ingredients | % w/w in stock |
|---|---|
| Phenyl ethanol | 18.1 |
| Thymol | 6.0 |
| Tetrahydrocurcumanoids | 3.0 |
| Benzoic acid | 12.0 |
| Ethanol | 52.8 |
| Grape seed oil | 9.0 |

### 4.3 Preservatives for Food and Beverage Products

Provided herein is a method for producing a preservative or antimicrobial effect (i.e., inhibiting the growth of microbes) in a food or beverage product comprising incorporating a preservative formulation as set forth herein into the product. Microbes, the growth of which are inhibited, include but are not limited to one or more of Aspergillus niger, Candida albicans, Staphylococcus aureus, Staphylococcus epidermidis, Escherichia coli, Pseudomonas aeruginosa, Enterococcus, Streptococcus species and Salmonella species (e.g. typhimurium, enteritidis) and Listeria monocytogenes.

Food and beverage products comprising the above ingredients include, but are not limited to, juices such as apple, tomato, orange, pineapple, cranberry and grapefruit juice (and mixtures thereof), fruit cocktails, soft drinks, sodas, punches, canned fruits and vegetables, related products such as fruit cocktail and apple sauce, salad dressings, condiments such as catsup, and baby foods.

In one set of non-limiting embodiments, the composition is a food preservative comprising the following components:

| Ingredients | % w/w stock |
|---|---|
| Biosecur ® | 15.29 |
| Cinnamon bark oil | 7.64 |
| Thymol | 0.3 |
| Alcohol | 76.45 |
| Benzoic acid | 0.3 |

In one set of non-limiting embodiments, the composition is a food preservative comprising the following components:

| Ingredients | % w/w final |
|---|---|
| Biosecur ® | 0.03 |
| Cinnamon bark oil | 0.01528 |
| Thymol | 0.0006 |
| Alcohol | 0.1528 |
| Benzoic acid | 0.0006 |

In one set of non-limiting embodiments, the composition is a food preservative comprising the following components:

| Ingredients | % w/w stock |
|---|---|
| Biosecur ® | 15.24 |
| Cinnamon bark oil | 7.62 |
| Thymol | 0.3 |
| Alcohol | 76.21 |
| Galangal oil | 0.3 |
| Benzoic acid | 0.3 |

In one set of non-limiting embodiments, the composition is a food preservative comprising the following components:

| Ingredients | % w/w final |
|---|---|
| Biosecur ® | 0.03 |
| Cinnamon bark oil | 0.0152 |
| Thymol | 0.0006 |
| Alcohol | 0.1524 |
| Galangal oil | 0.0006 |
| Benzoic acid | 0.0006 |

In one set of non-limiting embodiments, the composition is a food preservative comprising the following general components:

### Food Preservative—General Composition

| Ingredients | % w/w (Range) |
|---|---|
| Botanical extracts | 0.1-10 |
| Essential oil/ingredients | 3-10 |
| Organic acid | 1.0-5.0 |
| Alcohol | 10-85 |
| Odor masking carrier botanical oils | 0-10 |
| Odor masking essential oils | 0.0-0.10 |

In one set of non-limiting embodiments, the composition is a food preservative comprising the following components:

### Food Preservative RSV-6S

| | % w/w | Concentration (% w/w) in product |
|---|---|---|
| Ingredients | (stock) | containing 1% w/w preservative |
| Curcumin complex C3 | 0.49 | 0.005 |
| Cinnamon bark oil | 7.41 | 0.075 |
| Thymol | 4.94 | 0.05 |
| Benzoic acid | 2.47 | 0.025 |
| Ethanol | 83.49 | 0.845 |
| Sesame oil | 1.2 | 0.012 |

In one set of non-limiting embodiments, the composition is a food preservative comprising the following components:

### Food Preservative B-61A

| | % w/w | Concentration (% w/w) in product |
|---|---|---|
| Ingredients | (stock) | containing 1% w/w preservative |
| Citrus extract (Biosecur ®) | 9.52 | 0.04 |
| Cinnamon bark oil | 7.14 | 0.03 |
| Thymol | 4.76 | 0.02 |
| Benzoic acid | 2.38 | 0.01 |
| Ethanol | 11.9 | 0.05 |
| Olive oil | 59.5 | 0.25 |
| Sesame oil | 4.76 | 0.02 |

In one set of non-limiting embodiments, the composition is a beverage preservative comprising the following general components:

### Botani-que Beverage Preservative—General Composition

| Ingredients | % w/w (Range) |
|---|---|
| Botanical extracts | 0-15 |
| Essential oil/ingredients | 3-10 |
| Organic acid | 3.0-10 |
| Alcohol | 30-85 |
| Odor masking/Antioxidant botanical oils | 0-2.0 |
| Odor masking essential oils | 0.0-0.10 |

In one set of non-limiting embodiments, the composition is a beverage preservative comprising the following components:

### Beverage Preservative—20

| | % w/w | Concentration (% w/w) in product |
|---|---|---|
| Ingredients | (stock) | containing 1% w/w preservative |
| Thymol | 10 | 0.02 |
| Cinnamon bark oil | 5.0 | 0.01 |
| Benzoic acid | 10.0 | 0.02 |
| Alcohol | 67.5 | 0.135 |
| Vanilla oil | 7.6 | 0.015 |

In one set of non-limiting embodiments, the composition is a beverage preservative comprising the following components:

### Beverage Preservative—6-BR.1

| | % w/w | Concentration (% w/w) in product |
|---|---|---|
| Ingredients | (stock) | containing 1% w/w preservative |
| Thymol | 6.7 | 0.02 |
| Benzoic acid | 6.7 | 0.02 |
| Lemon extract | 3.3 | 0.01 |
| Alcohol | 83.3 | 0.25 |
| Resveratrol | 0.67 | 0.002 |

In one set of non-limiting embodiments, the composition is a beverage preservative comprising the following components:

### Beverage Preservative 6-BR

| Ingredients | % w/w |
|---|---|
| Thymol | 6.7 |
| Benzoic acid | 6.7 |
| Lemon extract | 3.3 |
| Alcohol | 82.63 |
| Resveratrol | 0.67 |

In one set of non-limiting embodiments, the composition is a beverage preservative comprising the following components:

### Beverage Preservative—6-CR.1

| | % w/w | Concentration (% w/w) in product |
|---|---|---|
| Ingredients | (stock) | containing 1% w/w preservative |
| Thymol | 3.3 | 0.01 |
| Benzoic acid | 6.7 | 0.02 |
| Lemon extract | 3.3 | 0.01 |
| Alcohol | 86.7 | 0.26 |
| Resveratrol | 0.67 | 0.002 |

In one set of non-limiting embodiments, the composition is a beverage preservative comprising the following components:

### Beverage Preservative 6-CR

| Ingredients | % w/w |
|---|---|
| Thymol | 6.7 |
| Benzoic acid | 6.7 |
| Lemon extract | 3.33 |
| Alcohol | 86.0.0 |
| Resveratrol | 0.67 |

In one set of non-limiting embodiments, the composition is a beverage preservative comprising the following components:

### Beverage Preservative—6-DR

| | % w/w | Concentration (% w/w) in product |
|---|---|---|
| Ingredients | (stock) | containing 1% w/w preservative |
| Thymol | 6.7 | 0.02 |
| Benzoic acid | 6.7 | 0.02 |
| Lemon extract | 6.7 | 0.02 |
| Alcohol | 79.33 | 0.24 |
| Resveratrol | 0.67 | 0.002 |

In one set of non-limiting embodiments, the composition is a beverage preservative comprising the following components:

### Beverage Preservative—6-BP.1

| | % w/w | Concentration (% w/w) in product |
|---|---|---|
| Ingredients | (stock) | containing 1% w/w preservative |
| Thymol | 6.7 | 0.02 |
| Benzoic acid | 6.7 | 0.02 |
| Lemon extract | 3.3 | 0.01 |
| Alcohol | 83.3 | 0.25 |
| Pomegranate extract | 0.67 | 0.002 |

In one set of non-limiting embodiments, the composition is a beverage preservative comprising the following components:

### Beverage Preservative 6-BP

| Ingredients | % w/w |
|---|---|
| Thymol | 6.7 |
| Benzoic acid | 6.7 |
| Lemon extract | 3.3 |
| Alcohol | 83.3.0 |
| Pomegranate Extract | 0.34 |

In one set of non-limiting embodiments, the composition is a beverage preservative comprising the following components:

### Beverage Preservative—6-CP.1

| | % w/w | Concentration (% w/w) in product |
|---|---|---|
| Ingredients | (stock) | containing 1% w/w preservative |
| Thymol | 3.3 | 0.01 |
| Benzoic acid | 6.7 | 0.02 |
| Lemon extract | 3.3 | 0.01 |
| Alcohol | 86.7 | 0.26 |
| Pomegranate extract | 0.67 | 0.002 |

In one set of non-limiting embodiments, the composition is a beverage preservative comprising the following components:

### Beverage Preservative 6-CP

| Ingredients | % w/w |
|---|---|
| Thymol | 6.7 |
| Benzoic acid | 6.7 |
| Lemon extract | 3.33 |
| Alcohol | 86.33 |
| Pomegranate extract | 0.34 |

In one set of non-limiting embodiments, the composition is a beverage preservative comprising the following components:

### Beverage Preservative—6-DP.1

| | % w/w | Concentration (% w/w) in product |
|---|---|---|
| Ingredients | (stock) | containing 1% w/w preservative |
| Thymol | 6.7 | 0.02 |
| Benzoic acid | 6.7 | 0.02 |
| Lemon extract | 6.7 | 0.02 |
| Alcohol | 80.0 | 0.24 |
| Pomegranate extract | 0.67 | 0.002 |

In one set of non-limiting embodiments, the composition is a beverage preservative comprising the following components:

### Beverage Preservative 6-DP

| Ingredients | % w/w |
|---|---|
| Thymol | 6.7 |
| Benzoic acid | 6.7 |
| Lemon extract | 6.7 |
| Alcohol | 79.66 |
| Pomogranate Extract | 0.34 |

In one set of non-limiting embodiments, the composition is a beverage preservative comprising the following components:

### Beverage Preservative—B 60

| | % w/w | Concentration (% w/w) in product |
|---|---|---|
| Ingredients | (stock) | containing 1% w/w preservative |
| Biosecur ® | 10 | 0.04 |
| Thymol | 5.0 | 0.02 |
| Cinnamon bark oil | 7.5 | 0.03 |
| Benzoic acid | 2.5 | 0.04 |
| Alcohol | 62.86 | 0.11 |
| Vanilla oil botanical oils | 8.57 | 0.015 |

In one set of non-limiting embodiments, the composition is a beverage preservative comprising the following components:

### Beverage Preservative—B 61

| | % w/w | Concentration (% w/w) in product |
|---|---|---|
| Ingredients | (stock) | containing 1% w/w preservative |
| Biosecur ® | 10 | 0.04 |
| Thymol | 5.0 | 0.02 |
| Cinnamon bark oil | 7.5 | 0.01 |
| Benzoic acid | 2.5 | 0.01 |
| Alcohol | 62.86 | 0.205 |
| Pomegranate oil | 8.57 | 0.015 |

In one set of non-limiting embodiments, the composition is a beverage preservative comprising the following components:

### Beverage Preservative—B 61.2

| | % w/w | Concentration (% w/w) in product |
|---|---|---|
| Ingredients | (stock) | containing 1% w/w preservative |
| Biosecur ® | 13.1 | 0.04 |
| Thymol | 6.7 | 0.02 |
| Benzoic acid | 6.7 | 0.02 |
| Alcohol | 66.7 | 0.20 |
| Lemon extract | 6.7 | 0.02 |

### 4.4 Disinfectants for Food

Disclosed herein are compositions that may be comprised in disinfectant compositions that may be used to reduce the number and/or growth of microbes on food products such as but not limited to vegetables, fruit, meat, poultry and fish. The disinfectant may be used by one or more of the original food producer or a food processor or the consumer. The food item may be washed, dipped, or soaked (for example, for between about 30 seconds and 1 hour or between about 30 seconds and 5 minutes) in the disinfectant composition, optionally followed by rinsing with water prior to consumption.

In certain embodiments the composition is a stock solution comprising the recited active agents and a suitable solvent. Non-limiting examples of suitable solvents include water, ethanol, glycerin, octoxyglycerin, and mixtures thereof.

As a non-limiting example, the stock disinfectant can be incorporated into the final product at a use level of between about 0.01 and 10 percent w/w.

Disclosed herein is a method of reducing the number and/or growth of microbes in or on a food product comprising exposing the food product to a disinfectant composition as set forth herein. Microbes, the numbers and/or growth of which are reduced, include but are not limited to one or more of Aspergillus niger, Candida albicans, Staphylococcus aureus, Staphylococcus epidermidis, Escherichia coli, Pseudomonas aeruginosa, Enterococcus, Streptococcus species, Salmonella species (e.g. typhimurium, enteritidis) and Listeria monocytogenes.

### 4.5 Topical Disinfectants

Disclosed herein are compositions that may be comprised in disinfectant compositions that may be used to topically to reduce the number and/or growth of microbes on the skin of a human or non-human animal. The disinfectant may be a wash solution, a lotion, a cream, or a splash and/or may be comprised in a wipe.

The disinfectant composition comprises the recited active agents and a suitable solvent. Non-limiting examples of suitable solvents include water, ethanol, glycerin, octoxyglycerin, and mixtures thereof.

Disclosed herein is a method of reducing the number and/or growth of microbes on the skin of a subject comprising applying to the skin a topical disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans, Aspergillus niger and Listeria monocytogenes.

### 4.6 Surface Disinfectants

Disclosed herein are compositions that may be comprised in disinfectant compositions that may be used to topically to reduce the number and/or growth of microbes on inanimate surfaces, for use in disinfecting, for example and not by way of limitation, hard surfaces such as floors, countertops, sinks, toilets, shower stalls, tables, furniture, car interiors, baby furniture, etc. as well as soft surfaces but non-absorbent surfaces such as leather or vinyl.

The disinfectant composition comprises the recited active agents and a suitable solvent. Non-limiting examples of suitable solvents include water, ethanol, glycerin, octoxyglycerin, and mixtures thereof.

Disclosed herein is a method of reducing the number and/or growth of microbes on an inanimate surface comprising applying to the surface a disinfectant composition as set forth herein. Microbes, the numbers and/or growth of which are reduced, include but are not limited to one or more of Staphylococcus aureus (including methicillin resistant Staphylococcus aureus), Staphylococcus epidermidis, Escherichia coli, Pseudomonas aeruginosa, Candida albicans, Aspergillus niger and Listeria monocytogenes.

The contents of the examples, including specific formulations, are hereby incorporated by reference into this detailed description.

In one set of non-limiting embodiments, the composition is a surface disinfectant comprising the following components:

| Ingredients | % w/w (range) |
|---|---|
| Citrus extract | 0.005-1.0 |
| Lemon oil | 0.05-0.5 |
| Thymol | 0.05-0.2 |
| Pine oil | 0.0-0.5 |
| Benzoic acid | 0.0-0.2 |
| Lactic acid/citric acid | 0.0-1.0 |
| Alkylpolyglucoside (Glucopon 425 N) | 0.5-3.0 |
| Solubilizer 611674 | 0.25-2.0 |
| Alcohol 3C | 5.0-20.0 |
| Diversaclean SRS | 0.5-2.0 |
| Volume adjusted to 100% with water | |

In certain specific non-limiting embodiments, the composition is a surface disinfectant comprising the following components:

### Specific Formulation-1

| Ingredients | % w/w |
|---|---|
| Lemon extract | 0.05 |
| Lemon oil | 0.1 |
| Thymol | 0.1 |
| Pine oil | 0.2 |
| Benzoic acid | 0.1 |
| Alkylpolyglucoside (Glucopon 425 N) | 1.0 |
| Solubilizer 611674 | 0.5 |
| Alcohol 3C | 5.0 |
| Diversaclean SRS | 1.0 |
| Water | 91.95 |

### Specific Formulation-2

| Ingredients | % w/w |
|---|---|
| Lemon extract | 0.05 |
| Lemon oil | 0.1 |
| Thymol | 0.1 |
| Pine oil | 0.2 |
| Lactic acid | 0.5 |
| Alkylpolyglucoside (Glucopon 425 N) | 1.0 |
| Solubilizer 611674 | 0.5 |
| Alcohol 3C | 5.0 |
| Diversaclean SRS | 1.0 |
| Water | 91.55 |

### Specific Formulation-3

| Ingredients | % w/w |
|---|---|
| Lemon extract | 0.05 |
| Lemon oil | 0.1 |
| Thymol | 0.1 |
| Pine oil | 0.2 |
| Benzoic acid | 0.005 |
| Lactic acid | 0.5 |
| Alkylpolyglucoside (Glucopon 425 N) | 1.0 |
| Solubilizer 611674 | 0.5 |
| Alcohol 3C | 5.0 |
| Diversaclean SRS | 1.0 |
| Water | 91.55 |

### Specific Formulation-4

| Ingredients | % w/w |
|---|---|
| Lemon extract | 0.3 |
| Lemon oil | 0.2 |
| Thymol | 0.1 |
| Pine oil | 0.2 |
| Lactic acid | 0.75 |
| Alkylpolyglucoside (Glucopon 425 N) | 1.0 |
| Solubilizer 611674 | 0.5 |
| Alcohol 3C | 5.0 |
| Diversaclean SRS | 1.0 |
| Water | 90.95 |

### 4.7 Oral Formulations

In certain embodiments, the compositions described herein are oral care compositions for use in reducing the number and/or growth of microbes in the mouth of a subject. Also provided is a method of reducing the number and/or growth of microbes in the mouth of a subject comprising applying the oral care composition to an inside surface of the mouth of the subject. As a non-limiting example, a stock oral care composition can be incorporated into the final product at a use level of between about 0.7 and 1.2% weight/weight (w/w) (such that the preservative constitutes between about 0.7 and 1.2% (w/w) of the final product), or at a use level of between about 1.0 and 2.0% w/w, at a use level of about 1.5% w/w.

In one set of non-limiting embodiments, the composition is an oral rinse comprising the following components:

| Ingredients | % w/w |
|---|---|
| Biosecur ® | 0.5-1.0 |
| Benzoic acid | 0.1-0.2 |
| Thymol | 0.05-0.1 |
| Alcohol | 0-10 |
| Tetrahydrocurcumanoid | 0-0.1 |
| Water | 70-90 |
| Sorbitol | 2.0-5.0 |
| Xylitol | 0.3-0.5 |
| Pluronic F127 | 0.1-0.5 |
| Menthol | 0.02-0.5 |
| Peppermint oil | 0.03-0.5 |

In one set of non-limiting embodiments, the composition is an oral rinse comprising the following components:

| Ingredients | % w/w |
|---|---|
| Biosecur ® | 0.5 |
| Benzoic acid | 0.1 |
| Thymol | 0.07 |
| Alcohol | 5.0 |
| Tetrahydrocurcumanoid | 0.05 |
| Water | 88.5 |
| Sorbitol | 5.0 |
| Xylitol | 0.5 |
| Pluronic F127 | 0.2 |
| Menthol | 0.03 |
| Peppermint oil | 0.05 |

### 4.8 Additional Ingredients

The compositions may contain additional components known in the art for use in personal care compositions.

In certain non-limiting embodiments, the composition does not contain a petroleum-derived component.

In certain non-limiting embodiments, the composition may comprise a polyethylene oxide (Polyox) hydrogel polymer which, without being bound to any particular theory, can help the skin retain moisture.

In certain non-limiting embodiments, the composition may comprise an anti-irritant component, for example a zinc salt, such as zinc gluconate, alpha bisabolol, aloe gel/leaf juice, and combinations thereof. Further non-limiting examples of zinc salts that may be used include zinc acetate, zinc butyrate, zinc citrate, zinc glycerate, zinc glycolate, zinc formate, zinc lactate, zinc picolinate, zinc propionate, zinc salicylate, zinc tartrate, zinc undecylenate, and combinations thereof.

For example, a composition disclosed herein may further comprise a thickening and/or gelling agent such as stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkoniurn bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. Gelling agents used in vehicles may be natural gelling agents such as natural gums, starches, pectins, agar and gelatin, and may be based on polysaccharides or proteins Examples include but are not limited to guar gum, xanthum gum, alginic acid (E400), sodium alginate (E401), potassium alginate (E402), ammonium alginate (E403), calcium alginate (E404, —polysaccharides from brown algae), agar (E406, a polysaccharide obtained from red seaweeds), carrageenan (E407, a polysaccharide obtained from red seaweeds), locust bean gum (E410, a natural gum from the seeds of the Carob tree), pectin (E440, a polysaccharide obtained from apple or citrus-fruit), and gelatin (E441, made by partial hydrolysis of animal collagen).

A composition as disclosed herein may optionally further comprise a surfactant. The surfactant may be an anionic surfactant, a cationic surfactant, an ampholytic surfactant, or a nonionic surfactant. Non-limiting examples of surfactants include polyethoxylates, fatty alcohols (e.g., ceteth-20 (a cetyl ether of polyethylene oxide having an average of about 20 ethylene oxide units) and other “BRIJ”™ nonionic surfactants available from ICI Americas, Inc. (Wilmington, Del.)), cocamidopropyl betaine, alkyl phenols, fatty acid esters of sorbitol, sorbitan, or polyoxyethylene sorbitan. Suitable anionic surfactants include ammonium lauryl sulfate and lauryl ether sulfosuccinate.

A composition as disclosed herein may optionally further comprise a silicone polymer, for example one or more than one polydimethylsiloxane polymer (Dow Corning 225 Silicone Fluid), dimethiconol fluid in dimethicone (Dow Corning 1403 Silicone Fluid), cyclomethicone and dimethicone copolyl (Dow Corning 3225C Silicone Fluid), silicone glycol (BASF 1066 DCG polyol), and combinations thereof.

A composition as disclosed herein may optionally further comprise one or more additives such as dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids, fruit acids and polycarboxylic acids; vitamins such as vitamin A, vitamin E and vitamin C; polyamino acids and salts, such as ethylenediamine tetraacidic acid (EDTA), preservatives such as German plus and DMDM hydantoin, and sunscreens such as aminobenzoic acid, 3,4 benzoic acid, arobenzone, cinoxate, diioxybenzone, homosalate, menthyl anthranilate, octocrylene, octyl methoxycinnamate, octyl salicylate, oxybenzoate, padimate 0, phenylbenzimidazole, sulfonic acid, sulisobenzone, titanium dioxide, trolamine salicylate and zinc oxide.

In certain non-limiting embodiments the composition may contain a cationic emulsifier selected from the group consisting of incroquat compounds such as (but not limited to) behenyltrimonium methosulfate in cetearyl alcohol (e.g., incroquat behenyl TMS and incroquat behenyl TMS 50 (Croda Inc., Edison, N.J.)), behenalkonium chloride and cetyl alcohol (e.g., Incroquat B-65 (Croda Inc., Edison, N.J.)), behenamido propyl ethyl dimonium ethosulfate and stearyl alcohol (Incroquat BES-35 S (Croda Inc., Edison, N.J.)), steralkonium chloride and cetearyl alcohol and PEG-40 Castor oil (e.g., Incroquat CR concentrate (Croda Inc., Edison, N.J.)), Incroquat CTC-30 (Croda Inc., Edison, N.J.), Incroquat DBM-90 (Croda Inc., Edison, N.J.), lncroquat 0-50 (Croda Inc., Edison, N.J.), lncroquat S-DQ-25 (Croda Inc., Edison, N.J.), Incroquat BA-85 (Croda Inc., Edison, N.J.), Incroquat WG-85 (Croda Inc., Edison, N.J.), as well as distearyldimonium chloride (e.g., VARISOFT® TA 100 (Essen-Degussa, Germany)), palmitamidopropyltrimonium chloride (e.g., VARISOFT® PATC (Essen-Degussa, Germany)), and cetearyl alcohol (and) palmitamidopropyltrimonium chloride (e.g., TEGO® Care CE 40).

### 4.9 Methods of Use

Disclosed herein are methods of inhibiting the growth of a microbe in a personal care formulation, comprising incorporating a composition, as described above, into said personal care formulation. In certain non-limiting embodiments, the microbe, the growth of which is inhibited, is a bacterium or yeast or fungus, for example, but not limited to, Staphylococcus aureus, Staphylococcus epidermidis, Enterococcus, Escherichia coli, Pseudomonas aeruginosa, Candida albicans, and Aspergillus niger. In certain non-limiting embodiments, the personal care formulation is an anionic formulation; in others, it is a cationic formulation. In certain non-limiting embodiments, growth is inhibited by at least about 10 percent or at least about 20 percent or at least about 30 percent or at least about 40 percent or at least about 50 percent. In certain non-limiting embodiments, the inhibition occurs over a period of up to about 3 months, or up to about 6 months, or up to about 12 months, or up to about 18 months, or up to about 24 months, or up to about 30 months.

### 5. EXAMPLE 1

### Efficacy of Portulaca Extract, Benzoic Acid, Essential Oils and Alcohol Against Various Pathogens

The formulations used in this study included portulaca extract combined with benzoic acid, essential oils and alcohol incorporated into an emulsion.

Methods:

The following method of testing for antimicrobial activity was used. 25 g of an emulsion containing 1-1.2% of a preservative composition was prepared. 0.9 gm aliquots of preservative containing emulsion were dispensed into several culture tubes. Culture tubes were divided into various groups and inoculated with 0.1 ml of the test organism (Staphylococcus aureus, Pseudomonas aeruginosa, Candida albicans (10⁸ cfu/ml) or Aspergillus niger (10⁷ cfu/ml)). After mixing, the tubes were incubated for 48 hours for S. aureus and P. aeruginosa and for 7 days for either C. albicans or A. niger. The cultures were then diluted serially with Drug Neutralizing Fluid (DNF) and 0.5 ml aliquots were plated on TSA and incubated for 24-48 hours (S. aureus, P. aeruginosa and C. albicans were incubated at 37° C. and A. niger at 30° C.). in the control group, the emulsion base without the preservatives was inoculated with each organism and processed similarly. The log 10 reduction in colony counts of the test group from the colony counts of control group was determined. The preservative compositions tested were as follows:

### Composition 1

| Ingredients | % w/w | % Range |
|---|---|---|
| Benzyl alcohol | 0.5 | 0.05-1.0 |
| Cinnamon Bark oil | 0.05 | 0.03-0.1 |
| Portulaca extract | 0.5 | 0.2-2.0 |
| Ethanol | 0.2 | 0.05-1.0 |

### Composition 1B

| Ingredients | % w/w | % Range |
|---|---|---|
| Benzyl alcohol | 0.5 | 0.05-1.0 |
| Cinnamon Bark oil | 0.05 | 0.03-0.1 |
| Portulaca extract | 0.5 | 0.2-2.0 |
| Benzoic acid | 0.1 | 0.1-0.5 |
| Ethanol | 0.2 | 0.05-1.0 |

### Composition 2

| Ingredients | % w/w | % Range |
|---|---|---|
| Benzyl alcohol | 0.5 | 0.05-1.0 |
| Thymol | 0.05 | 0.03-0.1 |
| Portulaca extract | 0.5 | 0.2-2.0 |
| Ethanol | 0.2 | 0.05-1.0 |

### Composition 2B

| Ingredients | % w/w | % Range |
|---|---|---|
| Benzyl alcohol | 0.5 | 0.05-1.0 |
| Thymol | 0.05 | 0.03-0.1 |
| Portulaca extract | 0.5 | 0.2-2.0 |
| Benzoic acid | 0.1 | 0.1-0.5 |
| Ethanol | 0.2 | 0.05-1.0 |

### Composition 3

| Ingredients | % w/w | % Range |
|---|---|---|
| Benzyl alcohol | 0.5 | 0.05-1.0 |
| Lemongrass oil | 0.05 | 0.03-0.1 |
| Portulacaa extract | 0.5 | 0.2-2.0 |
| Ethanol | 0.2 | 0.05-1.0 |

### Composition 3B

| Ingredients | % w/w | % Range |
|---|---|---|
| Benzyl alcohol | 0.5 | 0.05-1.0 |
| Lemongrass oil | 0.05 | 0.03-0.1 |
| Portulaca extract | 0.5 | 0.2-2.0 |
| Benzoic acid | 0.1 | 0.1-0.5 |
| Ethanol | 0.2 | 0.05-1.0 |

The results of the efficacy testing of the preservative compositions are shown in Table 1.

| | log reduction from control growth | | | |
|---|---|---|---|---|
| Composition | S. aureus | P. aeruginosa | C. albicans | A. niger |
| 1 | 4.5 | 5.0 | 3.0 | 1.70 |
| 1B | 5.0 | 5.0 | 4.6 | 5.5 |
| 2 | 3.50 | 5.0 | 2.5 | 1.80 |
| 2B | 6.0 | 6.0 | 5.0 | 6.0 |

Conclusion:

The antimicrobial efficacy of compositions 1B and 2B, which contain benzoic acid, was equal to or greater than compositions 1 and 2, respectively, which do not contain benzoic acid. The effect of the compositions comprising benzoic acid was significantly greater when tested against C. albicans and A. niger than the groups without benzoic acid.

### 6. EXAMPLE 2

### Efficacy of preservative Compositions Comprising Benzoic Acid, Alcohol Solvent, Essential Oils and/or Botanical Extracts Against Various Pathogens

Method.

The testing method used in this study is based on the guidelines issued by the Cosmetics, Toiletries, and Fragrance Association, Inc. (CTFA, 2001). 1.0% of preservative was added to 25 gms of the base. 0.9 gm aliquots were dispensed into culture tubes. The culture tubes were divided into groups and inoculated with 0.1 ml of the test organism (S. aureus and C. albicans (107 cfu/ml), and A niger (107 cfu/ml)). After mixing, the tubes were incubated for 48 hours for S. aureus and C. albicans, and 7 days for A. niger. The cultures were then diluted serially with Drug neutralizing fluid (DNF), and 0.5 ml aliquots were plated on TSA and incubated for 24-48 hours (S. aureus and C. albicans were incubated at 37° C., and A. niger at 30° C.). In the control group, the emulsion base without the preservatives was inoculated with each organism and was processed similarly. The log 10 reduction in colony counts of the test group from the colony counts of control group was determined.

The control base to which the preservative compositions were added comprised the following:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH 5.0 | |

Results.

The preservative formulations tested, and their efficacy against the test organisms are shown below:

TABLE 2
Efficacy against S. aureus

| | Efficacy (Log reduction |
|---|---|
| | from control growth) |
| Preservative | Test organism: S. aureus |
| Benzoic acid 0.1% + Ethanol 0.3% + | 0.5 |
| glycerin 0.6% | |
| Biosecur ® 0.5% + Glycerin 0.5% | 0.5 |
| Biosecur ® 0.5% + Benzoic acid 0.1% + | 3.6 |
| ethanol 0.1% + Glycerin0.2% | |
| Thymol .05% + Ethanol 0.3% + | 0.5 |
| glycerin 0.6% | |
| Cinnamon Bark oil 0.05% + Ethanol 0.3% + | 0.5 |
| glycerin 0.6% | |
| Biosecur ® 0.5% + Benzoic acid 0.1% + | 5.5 |
| thymol 0.05% + ethanol 0.1% | |
| Biosecur ® 0.5% + Benzoic acid 0.1% + | 6.0 |
| Cinnamon Bark oil 1 0.05% + ethanol 0.1% | |
| Lemon extract 0.5% + Benzoic acid 0.1% + | 5.5 |
| thymol 0.05% + ethanol 0.1% | |
| Phenyl ethanol 0.3% + 0.7% glycerin | 0.5 |
| Benzyl alcohol 0.3% + glycerin 0.7% | 0.4 |
| Benzoic acid 0.1% + Phenyl ethanol 0.3% + | 4.6 |
| glycerin 0.6% | |
| Biosecur ® 0.5% + Benzoic acid 0.1% + | 4.6 |
| Phenylethanol 0.4% | |
| Phenylethanol 0.5% + Thymol 0.05% + | 0.8 |
| Glycerin 0.45% | |
| Biosecur ® 0.5% + Benzoic acid 0.1% + | 6.0 |
| Thymol 0.05% + Phenyl ethanol 0.35% | |

Conclusion:

benzoic acid exhibits synergistic activity when combined with citrus extracts and phenyl ethanol. A combination of benzoic acid, citrus extract, phenyl ethanol and essential oils further enhances the activity. Benzoic acid also shows enhanced activity when combined with citrus extract, essential oil and ethanol.

TABLE 3
Efficacy against A. niger

| | Efficacy (Log reduction |
|---|---|
| | from control growth) |
| Preservative | Test organism: A, niger |
| Biosecur ® 0.5% | 0.7 |
| Phenyl ethanol 0.3% + glycerin 0.7% | 0.5 |
| Benzyl alcohol 0.3% + glycerin 0.7% | 0.4 |
| Biosecur ® 0.5% + Benzoic acid 0.1% + | 3.05 |
| alcohol 0.1% | |
| Biosecur ® 0.5% + Benzoic acid 0.1% + | 6.5 |
| thymol 0.05% Alcohol 0.1% | |
| Biosecur ® 0.5% + Lactic acid 0.1% + | 1.0 |
| thymol 0.05% + ethanol 0.1% | |
| Benzoic acid 0.1% + Phenyl ethanol 0.3% + | 4.6 |
| glycerin 0.6% | |
| Benzoic acid 0.1% + Phenyl ethanol 0.3% + | 5.0 |
| Thymol 0.05% + glycerin 0.1% | |
| Benzoic acid 0.1% + Phenyl ethanol 0.3% + | 6.0 |
| Biosecur ® 0.5% + Thymol 0.05% + | |
| glycerin 0.1% | |
| Benzoic acid 0.1% + Phenyl ethanol 0.3% + | 6.0 |
| Biosecur ® 0.5% + Cinnamon bark oil 0.05% | |
| glycerin 0.05% | |
| Lemon extract 0.5% + Thymol 0.05% + | 6.0 |
| Benzoic acid 0.1% + Ethanol 0.35% | |
| Lemon extract 0.5% + Thymol 0.05% + | 6.0 |
| Benzoic acid 0.1% + Phenyl ethanol 0.35% | |
| Lemon extract 0.5% + Thymol 0.05% + | 6.0 |
| Benzoic acid 0.1% + Benzyl alcohol 0.35% | |
| Portulaca extract 0.5% + Benzoic acid 0.1% + | 6.5 |
| thymol 0.05% + Phenyl ethanol 0.35% | |
| Portulaca extract 0.5% + Benzoic acid 0.1% + | 6.5 |
| thymol 0.05% + Ethanol 0.35% | |

Conclusion:

Preservative compositions comprising benzoic acid, botanical extract and essential oil in an alcoholic solvent show significant efficacy against A. niger.

TABLE 4
Efficacy against A. niger

| | Efficacy (Log reduction |
|---|---|
| | from control growth) |
| Preservative | Test organism: A, niger |
| Lactic acid 0.1% + Ethanol 0.3% + | 0.5 |
| glycerin 0.6% | |
| Biosecur ® 0.5% + Lactic acid 0.1% + | 0.5 |
| ethanol 0.1% + Glycerin 0.2% | |
| Biosecur ® 0.5% + Lactic acid 0.1% + | 0.7 |
| thymol 0.05% + ethanol 0.1% | |
| Biosecur ® 0.5% + Lactic acid 0.1% + | 0.8 |
| thymol 0.05% + Phenyl ethanol 0.35% | |
| Lemon extract 0.5% + Lactic acid 0.1% + | 0.8 |
| thymol 0.05% + Phenyl ethanol 0.35% | |
| Portulaca extract 0.5% + Lactic acid | 0.8 |
| 0.1% + thymol 0.05% + Phenyl ethanol | |
| 0.35% | |

Conclusion:

In these studies, lactic acid did not enhance the activity of formulations comprising botanical extract, essential oil and solvent against A. niger. Furthermore, lactic acid has an unpleasant odor which may not be suitable for use in certain personal care products.

### 7. EXAMPLE 3

### Efficacy of Preservative Compositions Comprising Benzoic Acid, Alcohol Solvent, Essential Oils and/or Botanical Extracts Against Various Pathogens

The antimicrobial effect of the following preservative compositions were tested against A. niger:

### General Preservative Composition

1)

| Ingredients | % w/w | Range % w/w |
|---|---|---|
| Alcohol (ethanol, phenyl ethanol | 18.75 | 5-45 |
| and/or benzyl alcohol) | | |
| Benzoic acid | 12.5 | 2-20 |
| Citrus extract | 62.5 | 20-70 |
| Essential oil | 6.25 | 1-10 |

2)

| Ingredients | % w/w | Range % w/w |
|---|---|---|
| Alcohol (ethanol, phenyl ethanol | 23.52 | 5.0-45.0 |
| and/or benzyl alcohol) | | |
| Benzoic acid | 11.76 | 2-20 |
| Botanical extract | 58.82 | 20-70 |
| Essential oil/ingredients | 5.88 | 1-10 |

3)

| Ingredients | % w/w | Range % w/w |
|---|---|---|
| Alcohol (ethanol, phenyl ethanol | 71.52 | 30.0-80.0 |
| and/or benzyl alcohol) | | |
| Benzoic acid | 14.20 | 2-20 |
| Botanical extract | 7.14 | 5-50 |
| Essential oil/ingredients | 7.14 | 1-10 |

The use level of all the formulation is 0.7-1.0

### SHL)

| Ingredients | Use concentration % w/w | % w/w Stock |
|---|---|---|
| Phenyl ethanol | 0.5 | 71.52 |
| Tetrahydrocurcumanoids | 0.05 | 7.14 |
| Benzoic acid | 0.1 | 14.20 |
| Thymol | 0.05 | 7.14 |

### SHL1)

| Ingredients | Use concentration % w/w | % w/w Stock |
|---|---|---|
| Phenyl ethanol | 0.3 | 42.81 |
| Tetrahydrocurcumanoids | 0.05 | 7.14 |
| Benzoic acid | 0.1 | 14.20 |
| Portulaca extract | 0.2 | 28.57 |
| Thymol | 0.05 | 7.14 |

### SHL2)

| Ingredients | Use concentration % w/w | % w/w Stock |
|---|---|---|
| Phenyl ethanol | 0.4 | 57.14 |
| Tetrahydrocurcumanoids | 0.05 | 7.14 |
| Benzoic acid | 0.1 | 14.20 |
| Portulaca extract | 0.1 | 14.28 |
| Thymol | 0.05 | 7.14 |

### BB1)

| Ingredients | Use concentration % w/w | % w/w Stock |
|---|---|---|
| Phenyl ethanol | 0.3 | 31.57 |
| Biosecur | 0.5 | 52.63 |
| Benzoic acid | 0.1 | 10.52 |
| Thymol | 0.05 | 5.26 |

### BB2)

| Ingredients | Use concentration % w/w | % w/w Stock |
|---|---|---|
| Biosecur | 0.5 | 66.66 |
| Benzoic acid | 0.1 | 13.33 |
| Thymol | 0.05 | 6.66 |
| Alcohol | 0.1 | 13.33 |

### BB2A)

| Ingredients | Use concentration % w/w | % w/w Stock |
|---|---|---|
| Biosecur | 0.7 | 70.00 |
| Benzoic acid | 0.1 | 10.00 |
| Thymol | 0.05 | 5.00 |
| Alcohol | 0.15 | 15.00 |

### BB3)

| Ingredients | Use concentration % w/w | % w/w Stock |
|---|---|---|
| Phenyl ethanol | 0.3 | 31.57 |
| Benzoic acid | 0.1 | 10.52 |
| Thymol | 0.05 | 5.26 |
| Lemon extract | 0.5 | 52.63 |

### PB1)

| Ingredients | Use concentration % w/w | % w/w Stock |
|---|---|---|
| Alcohol | 0.1 | 13.33 |
| Portulaca extract | 0.5 | 66.66 |
| Benzoic acid | 0.1 | 13.33 |
| Cinnamon oil | 0.05 | 6.66 |

### PB2)

| Ingredients | Use concentration % w/w | % w/w Stock |
|---|---|---|
| Phenyl ethanol | 0.3 | 31.57 |
| Portulaca extract | 0.5 | 52.63 |
| Benzoic acid | 0.1 | 10.52 |
| Cinnamon oil | 0.05 | 5.26 |

### E4)

| Ingredients | Use concentration % w/w | % w/w Stock |
|---|---|---|
| Phenyl ethanol | 0.3 | 42.85 |
| Echinacea extract | 0.15 | 21.42 |
| Benzoic acid | 0.1 | 14.28 |
| Thymol | 0.05 | 7.14 |
| Alcohol | 0.1 | 14.28 |

The antimicrobial effect of the above preservative compositions against A. niger are shown in Table 5.

TABLE 5

| | Log₁₀ reduction from control | |
|---|---|---|
| Group | Growth A. niger (7 day) | Use level (%) |
| BB1 | 6.0 | 1.0 |
| BB2 | 6.5 | 1.0 |
| BB3 | 5.0 | 1.0 |
| PB1 | 6.6 | 1.0 |
| PB2 | 6.5 | 1.0 |

Control growth was 1×10⁷-5×10⁷

### 8. EXAMPLE 4

### Efficacy of Preservative Compositions Comprising Benzoic Acid, Alcohol Solvent, Essential Oils and Botanical Extract Against Aspergillus niger

The antimicrobial effect of the following preservative compositions were tested against A. niger:

### FP2

| Ingredients | % w/w |
|---|---|
| Biosecur | 15.2 |
| Cinnamon bark oil | 7.6 |
| Thymol | 0.3 |
| Ethanol | 76 |
| Benzoic acid | 0.3 |
| Galan gal oil | 0.3 |
| Use level 0.1-0.2% | |

### Method

Apple juice containing 0.1-0.2% of FP2 preservative were prepared. 5.0 ml of the juice was distributed in culture tubes and inoculated with 10² cfu of A. niger and incubated for 21 days. Microbial growth in the juice was noted. As a control, plain juice without preservative was inoculated with the same culture.

### Results

The antimicrobial effect of the above preservative composition against A. niger is shown in Table 6.

TABLE 6
Growth of A. niger in inoculated Juice

| | Day 2 | Day 7 | Day 14 | Day 21 | Day 28 |
|---|---|---|---|---|---|
| Control | ++ | +++ | +++ | +++ | +++ |
| FP2 | −− | −− | −− | −− | −− |

Legend:
+++: Heavy growth
++: Medium growth
−−: No growth

### 9. EXAMPLE 5

### Efficacy of Preservative Compositions Comprising Benzoic Acid, Alcohol Solvent, Essential Oils and Botanical Extract Against Aspergillus niger

The antimicrobial effect of the following preservative compositions were tested against A. niger:

### FP1

| Ingredients | Use concentration % w/w | % w/w Stock |
|---|---|---|
| Biosecur ® | 0.03 | 35.29 |
| Cinnamon bark oil | 0.01528 | 7.64 |
| Thymol | 0.0006 | 0.3 |
| Alcohol | 0.1528 | 76.45 |
| Benzoic acid | 0.0006 | 0.3 |

### SHL

| Ingredients | Use concentration % w/w | % w/w Stock |
|---|---|---|
| Phenyl ethanol | 0.5 | 71.52 |
| Tetrahydrocurcumanoids | 0.05 | 7.14 |
| Benzoic acid | 0.1 | 14.20 |
| Thymol | 0.05 | 7.14 |

### SHL1

| Ingredients | Use concentration % w/w | % w/w Stock |
|---|---|---|
| Phenyl ethanol | 0.3 | 42.81 |
| Tetrahydrocurcumanoids | 0.05 | 7.14 |
| Benzoic acid | 0.1 | 14.20 |
| Portulaca extract | 0.2 | 28.57 |
| Thymol | 0.05 | 7.14 |

### SHL2

| Ingredients | Use concentration % w/w | % w/w Stock |
|---|---|---|
| Phenyl ethanol | 0.4 | 57.14 |
| Tetrahydrocurcumanoids | 0.05 | 7.14 |
| Benzoic acid | 0.1 | 14.20 |
| Portulaca extract | 0.1 | 14.28 |
| Thymol | 0.05 | 7.14 |

### Cosmetic Preservative 2

| Ingredients | % w/w in stock |
|---|---|
| Phenyl ethanol | 18.1 |
| Thymol | 6.0 |
| Tetrahydrocurcumanoids | 3.0 |
| Benzoic acid | 12.0 |
| Ethanol | 54.8 |
| Safflower oil | 6.0 |
| Vanilla oil | 0.125 |

### Cosmetic Preservative 4

| Ingredients | % w/w in stock |
|---|---|
| Phenyl ethanol | 18.1 |
| Thymol | 6.0 |
| Tetrahydrocurcumanoids | 3.0 |
| Benzoic acid | 12.0 |
| Ethanol | 54.8 |
| Safflower oil | 6.0 |
| Vanilla oleoresin | 0.125 |
| Pomegranate extract | 0.062 |

### Cosmetic Preservative 6

| Ingredients | % w/w in stock |
|---|---|
| Phenyl ethanol | 18.1 |
| Thymol | 6.0 |
| Tetrahydrocurcumanoids | 3.0 |
| Benzoic acid | 12.0 |
| Ethanol | 52.8 |
| Grape seed oil | 9.0 |

### Method

The following method of testing for antimicrobial activity was used. 25 g of an emulsion containing 1-1.2% of a preservative composition was prepared. 0.9 gm aliquots of preservative containing emulsion were dispensed into several culture tubes. Culture tubes were divided into various groups and inoculated with 0.1 ml of the test organism Aspergillus niger (10⁷ cfu/ml). After mixing, the tubes were incubated for 7 days. The cultures were then diluted serially with Drug Neutralizing Fluid (DNF) and 0.5 ml aliquots were plated on TSA and incubated for 24-48 hours at 30° C. In the control group, the emulsion base without preservative was inoculated with each organism and processed similarly. The log 10 reduction in colony counts of the test group from the colony counts of control group was determined.

### Results

The antimicrobial effect of the above preservative compositions against A. niger are shown in Table 7.

### Efficacy Against A. niger

### Efficacy (Log Reduction from Control Growth)

| Preservative | Test organism: A. niger |
|---|---|
| FP1 | 5.5 |
| SHL | 5.0 |
| SHL1 | 5.2 |
| SHL2 | 5.0 |

### 10. EXAMPLE 6

### Minimum Inhibitory Concentration (MIC) of Cosmetic Preservative Composition PB-V1 Comprising Botanical Extract, Benzyl Alcohol, Essential Oil, Benzoic Acid, Alcohol Solvent, and Vanilla Oleo Resin Against Various Microbes

The antimicrobial effect of cosmetic preservative composition PB-V1 was tested against various microbes:

### Cosmetic Preservative PB-V1

| Ingredients | % w/w |
|---|---|
| Biosecur ® A 520 D | 15.0 |
| Benzyl alcohol | 30.0 |
| Thymol | 5.0 |
| Benzoic acid | 10 |
| Alcohol | 40.0 |
| Vanilla oleo resin | 1.25 |

### Method of MIC Test

Various concentrations of the preservative was a dded to 5.0 ml Tripticase Soy broth and inoculsated with 0.1 ml of 10⁶ cfu/ml of bacteria After 24-48 hours the turbidity of solution was noted. The lowest concentration which does not show turbidity is the MIC concentration.

### Results

Control growth was 1×10⁷ to 5×10⁷

The MIC of cosmetic preservative composition PB-V1 on various microbes was as follows:

| Microorganism & ATCC number | PB-V1 (ppm) |
|---|---|
| S. aureus ATCC 6538 | 15.0 |
| E. coli ATCC 35218 | 125.0 |
| P. aeroginosa ATCC 9027 | 125.0 |
| A. niger ATCC 16404 | 62.5 |
| C. albicans ATCC 10231 | 62.5 |

### 11. EXAMPLE 7

### Antimicrobial Efficacy of Cosmetic Preservative Composition P-V Comprising Botanical Extract, Benzyl Alcohol, Essential Oil, Benzoic Acid, Alcohol Solvent, and Vanilla Oleo Resin and Against Various Microbes

The antimicrobial effect of cosmetic preservative composition P-V was tested against various microbes:

### Cosmetic Preservative P-V

| Ingredients | % w/w |
|---|---|
| Biosecur ® A 520 D | 15.0 |
| Benzyl alcohol | 30.0 |
| Thymol | 5.0 |
| Benzoic acid | 10 |
| Alcohol | 40.0 |
| Vanilla oleo resin | 1.25 |

The control lotion to which the preservative was added comprised the following ingredients:

| Ingredient | % w/w |
|---|---|
| Water | 78.0 |
| Propylene glycol | 2.0 |
| Mineral oil | 9.5 |
| Ceteareth 25 | 2.5 |
| Cetearyl Alcohol | 4.0 |
| Glyceryl stearate | 2.0 |
| Monoglyceryl citrate | 2.0 |
| pH 5.0 | |

### Method

The antimicrobial efficacy of each treatment group described below was tested against the following microbes:

### Test Organisms (ATCC)

Aspergillus niger (16404)
Pseudomonas aeruginosa (9027)
Candida albicans (10231)
Escherichia coli (11229)

The testing method used in this study is based on the guidelines issued by the Cosmetics, Toiletries, and Fragrance Association, Inc. [CTFA, 2001]. Briefly 1.0% of preservative was added to 25 g of the control emulsion. 0.9 g aliquots were dispensed into several culture tubes. The tubes were divided into three groups and inoculated with 0.1 ml of the test organism (bacteria and C. albicans (10⁸ cfu/ml) and A. niger (10⁷ cfu/ml), as described below. After mixing, the tubes were incubated for 48 hours for bacteria and C. albicans and 7 days for A. niger respectively. The cultures were then diluted serially with Drug Neutralizing Fluid (DNF) and 0.5 ml aliquots were plated on TSA and incubated for 24-48 hours (bacteria and C. albicans were incubated at 37° C. and A. niger at 30° C.). In the control group, the emulsion base without the preservatives was inoculated with each organism and was processed similarly. The log₁₀ reduction in colony counts of the test group from the colony counts of control group was determined.

After mixing, tubes from all the groups were also incubated for 2, 7, 14, 21 and 28 days for all microorganisms. The cultures were then diluted serially with Drug Neutralizing Fluid (DNF), and a 0.5 ml aliquot from each tube was plated on TSA and incubated for 24-48 hours (S. aureus, P. aeruginosa and C. albicans were incubated at 37° C. and A. niger at 30° C.). In the control group, the lotion base without the preservative was inoculated with each microorganism and was processed similarly. The log₁₀ reduction in colony counts of the test group from the colony counts of the control group was determined.

Group 1: 25 ml portions of the samples were inoculated with 0.1 ml microorganism suspension. The initial microorganism count was approximately 10⁸ cfu/ml (for A. niger 10⁷ cfu/ml was used) and stirred.
Group 2: Another set of samples were kept at room temperature for 3 months and then inoculated with microorganism as described above for Group 1.
Group 3: The preservative was stored at 40° C. for 2 weeks (accelerated aging), and then incorporated into the control lotion and inoculated with microorganisms as described above for Group 1.

### Results

### Preservative Efficacy of Botani-que Cosmetic Preservative (P-V)

### (Colony Counts (CFU)/ml)

### Group 1

| | | S. aureus | P. aeruginosa | C. albicans | A. niger |
|---|---|---|---|---|---|
| | | ATCC | ATCC | ATCC | ATCC |
| | | 6538 | 15442 | 10231 | 16404 |
| Base Cream | | 5 × 10⁶ | 5 × 10⁶ | 5 × 10⁶ | 1 × 10⁶ |
| (Control) | | | | | |
| 2 | day | <10 | <10 | <10 | Not Done |
| 7 | day | <10 | <10 | <10 | <10 |
| 14 | day | <10 | <10 | <10 | <10 |
| 21 | day | <10 | <10 | <10 | <10 |
| 28 | day | <10 | <10 | <10 | <10 |

### Group 2

| | | S. aureus | P. aeruginosa | C. albicans | A. niger |
|---|---|---|---|---|---|
| | | ATCC | ATCC | ATCC | ATCC |
| | | 6538 | 15442 | 10231 | 16404 |
| Base Cream | | 5 × 10⁶ | 5 × 10⁶ | 5 × 10⁶ | 1 × 10⁶ |
| (Control) | | | | | |
| 2 | day | <10 | <10 | <10 | Not Done |
| 7 | day | <10 | <10 | <10 | <10 |
| 14 | day | <10 | <10 | <10 | <10 |
| 21 | day | <10 | <10 | <10 | <10 |
| 28 | day | <10 | <10 | <10 | <10 |

### Group 3

| | | S. aureus | P. aeruginosa | C. albicans | A. niger |
|---|---|---|---|---|---|
| | | ATCC | ATCC | ATCC | ATCC |
| | | 6538 | 15442 | 10231 | 16404 |
| Base Cream | | 5 × 10⁶ | 5 × 10⁶ | 5 × 10⁶ | 1 × 10⁶ |
| (Control) | | | | | |
| 2 | day | <10 | <10 | 1.2 × 10² | Not Done |
| 7 | day | <10 | <10 | <10 | <10 |
| 14 | day | <10 | <10 | <10 | <10 |
| 21 | day | <10 | <10 | <10 | <10 |
| 28 | day | <10 | <10 | <10 | <10 |

### Conclusion

Botani-que cosmetic preservative P-V was an effective antimicrobial preservative against the microorganisms tested. Cosmetic preservative P-V is odorless and colorless and can be used in personal care products ranging from pH 4-8.

### 12. EXAMPLE 8

### Antimicrobial Efficacy of Botani-que Food Preservative Composition RSV-6-S in Tomato Sauce

The antimicrobial efficacy of the following food preservative composition was tested:

Food preservative RSV-6S
Use level 0.5%-1.0% w/w

### Composition of RSV-6S

| Ingredients | % w/w | Range (% w/w) |
|---|---|---|
| Curcumin complex (C3 curcumin) | 0.49 | 0.3-0.6 |
| Thymol | 4.94 | 4.0-8.0 |
| Cinnamon bark oil | 7.41 | 5.0-10.0 |
| Benzoic acid | 2.47 | 2.0-4.0 |
| Alcohol | 83.5 | 50-85 |
| Sesamee oil | 1.19 | 0.5-2.0 |

### Method

Challenge tests in Ragu Tomato Sauce: Sauce containing 0.8% w/w beverage preservative RSV-6S was distributed in several culture tubes. The culture tubes were inoculated with various organisms to a final concentration of 10⁵ cfu/ml of sauce and incubated at 37° C. in an incubator. For control, sauce containing 0.8% w/w saline was used. At various intervals, 3 tubes from each group were removed and diluted with drug inactivating media. After serial dilution, aliquots were plated on TSA plate and incubated at 37° C. to determine the colony counts.

### Results

No taste difference in the tomato sauce was detectable between sauce that comprised the preservative and the control sauce without preservative.

The antimicrobial efficacy of food preservative RSV-6S against various microorganisms after 0, 7, 14, 21 and 28 days incubation is shown below.

| | | E. Coli | P. aeroginosa | S. aureus | C. albicans | A. niger |
|---|---|---|---|---|---|---|
| | | ATCC | ATCC | ATCC | ATCC | ATCC |
| | | 35218 | 9027 | 6538 | 10231 | 16404 |
| 0 | days | 10⁵ | 5.2 × 10⁵ | 4.2 × 10⁵ | 2.0 × 10⁶ | 1.0 × 10⁶ |
| 7 | days | <10 | <10 | <10 | <10 | <10 |
| 14 | days | <10 | <10 | <10 | <10 | <10 |
| 21 | days | <10 | <10 | <10 | <10 | <10 |
| 28 | days | <10 | <10 | <10 | <10 | <10 |

### 13. EXAMPLE 9

### Antimicrobial Efficacy of Botani-que Beverage Preservative Compositions 20, 6-DR, and B-60 in Clear Apple Juice

The antimicrobial efficacy of beverage preservatives 20, 6-DR, and B-60 in clear apple juice was tested. The three compositions tested have the following formulations:

### Beverage Preservative 20

| Ingredients | % w/w |
|---|---|
| Thymol | 10 |
| Cinnamon bark oil | 5.0 |
| Benzoic acid | 10.0 |
| Alcohol | 67.5 |
| Vanilla oil | 7.6 |

### Beverage Preservative 6-DR

| Ingredients | % w/w |
|---|---|
| Thymol | 6.7 |
| Benzoic acid | 6.7 |
| Lemon extract | 6.7 |
| Alcohol | 79.33 |
| Resveratrol | 0.67 |

### Beverage Preservative 6-BR

| Ingredients | % w/w |
|---|---|
| Thymol | 6.7 |
| Benzoic acid | 6.7 |
| Lemon extract | 3.3 |
| Alcohol | 82.63 |
| Resveratrol | 0.67 |

### Beverage Preservative 6-CR

| Ingredients | % w/w |
|---|---|
| Thymol | 6.7 |
| Benzoic acid | 6.7 |
| Lemon extract | 3.33 |
| Alcohol | 86.0.0 |
| Resveratrol | 0.67 |

### Beverage Preservative B-60

| Ingredients | % w/w |
|---|---|
| Biosecur ® | 10 |
| Thymol | 5.0 |
| Cinnamon bark oil | 7.5 |
| Benzoic acid | 2.5 |
| Alcohol | 62.86 |
| Vanilla oil/botanical oils | 8.57 |

### Beverage Preservative 6-DP

| Ingredients | % w/w |
|---|---|
| Thymol | 6.7 |
| Benzoic acid | 6.7 |
| Lemon extract | 6.7 |
| Alcohol | 79.66 |
| Pomogranate Extract | 0.34 |

### Beverage Preservative 6-BP

| Ingredients | % w/w |
|---|---|
| Thymol | 6.7 |
| Benzoic acid | 6.7 |
| Lemon extract | 3.3 |
| Alcohol | 83.3.0 |
| Pomogranate Extract | 0.34 |

### Beverage Preservative 6-CP

| Ingredients | % w/w |
|---|---|
| Thymol | 6.7 |
| Benzoic acid | 6.7 |
| Lemon extract | 3.33 |
| Alcohol | 86.33 |
| Pomogranate extract | 0.34 |

### Method

Challenge Test: Apple juice containing 0.2% of beverage preservative 20, 6-DR or B-60 was distributed in several culture tubes. The culture tubes were inoculated with various organisms to a final concentration of 10² cfu/ml of apple juice. Organism growth in the apple juice comprising preservative (T) and control apple juice without preservative (C) was determined.

### Results

The antimicrobial efficacy of apple juice containing 0.3% beverage preservative 20, 6-DR and B-60 is shown below:

| | S. aureus | | E. coli | | A. niger | | C. albicans | |
|---|---|---|---|---|---|---|---|---|
| Days | C | T | C | T | C | T | C | T |
| 1 | + | − | + | − | + | − | − | − |
| 2 | ++ | − | ++ | − | ++ | − | + | − |
| 3 | +++ | − | +++ | − | +++ | − | + | − |
| 4 | +++ | − | +++ | − | +++ | − | ++ | − |
| 7 | +++ | − | +++ | − | +++ | − | +++ | − |
| 14 | +++ | − | +++ | − | +++ | − | +++ | − |
| 28 | +++ | − | +++ | − | +++ | − | +++ | − |

+ Growth
+++ Heavy Growth
− No growth

All 3 preservative showed the same result.

### Conclusion

Beverage preservative 20, 6-DR and B-60 are effective for up to 28 days.

Various publications are cited herein, the contents of which are hereby incorporated by reference in their entireties.

We claim:

1. A composition comprising an effective antimicrobial amount of
(i) benzoic acid in an amount between about 0.0001 and 20 percent weight/weight;
(ii) a botanical extract selected from the group consisting of Biosecur®, citrus extract, portulaca extract, tetrahydocurcumanoids (THC) and derivatives thereof, echinacea extract, and combinations thereof, in an amount between about 0.005 and 70 percent weight/weight;
(iii) an essential oil, or active constituent thereof, selected from the group consisting cinnamon oil, lemon oil, thyme oil, galangal oil, thymol, cinnamaldehyde and combinations thereof, in an amount between about 0.0001 and 10 percent weight/weight; and
(iv) an alcohol solvent selected from the group consisting of phenyl ethanol, ethanol, benzyl alcohol and combinations thereof, in an amount between about 0.01 and 80 percent weight/weight.

2. The composition of claim 1, wherein the composition comprises
(i) benzoic acid in an amount between about 0.01 and 0.2 percent weight/weight;
(ii) Biosecur® in an amount between about 0.05 and 1 percent weight/weight;
(iii) thyme oil, thymol or combinations thereof, in an amount between about 0.01 and 1 percent weight/weight; and
(iv) an alcohol solvent selected from the group consisting of phenyl ethanol, ethanol, benzyl alcohol and combinations thereof, in an amount between about 0.01 and 1 percent weight/weight.

3. The composition of claim 1, wherein the composition comprises
(i) benzoic acid in an amount between about 2 and 20 percent weight/weight;
(ii) Biosecur® in an amount between about 20 and 70 percent weight/weight;
(iii) thyme oil, thymol or combinations thereof, in an amount between about 1 and 10 percent weight/weight; and
(iv) an alcohol solvent selected from the group consisting of phenyl ethanol, ethanol, benzyl alcohol and combinations thereof, in an amount between about 5 and 45 percent weight/weight.

4. The composition of claim 1, wherein the composition comprises
(i) benzoic acid in an amount between about 0.0001 and 0.1 percent weight/weight;
(ii) Biosecur® in an amount between about 0.005 and 1 percent weight/weight;
(iii) an essential oil, or active constituent thereof, selected from the group consisting cinnamon oil, thyme oil, galangal oil, thymol, cinnamaldehyde and combinations thereof, in an amount between about 0.0001 and 0.5 percent weight/weight; and
(iv) an alcohol solvent selected from the group consisting of phenyl ethanol, ethanol, benzyl alcohol and combinations thereof, in an amount between about 0.01 and percent weight/weight.

5. The composition of claim 1, wherein the composition comprises
(i) benzoic acid in an amount between about 0.01 and 1 percent weight/weight;
(ii) Biosecur® in an amount between about 5 and 30 percent weight/weight;
(iii) an essential oil, or active constituent thereof, selected from the group consisting cinnamon oil, thyme oil, galangal oil, thymol, cinnamaldehyde and combinations thereof, in an amount between about 0.01 and 10 percent weight/weight; and
(iv) an alcohol solvent selected from the group consisting of phenyl ethanol, ethanol, benzyl alcohol and combinations thereof, in an amount between about 20 and 80 percent weight/weight.

6. The composition of claim 1, wherein the composition comprises
(i) benzoic acid in an amount between about 0.01 and 0.2 percent weight/weight;
(ii) a botanical extract selected from the group consisting of portulaca extract, tetrahydocurcumanoids (THC) and derivatives thereof, and combinations thereof, in an amount between about 0.01 and 1 percent weight/weight;
(iii) thyme oil, thymol, or combinations thereof, in an amount between about 0.01 and 1 percent weight/weight; and
(iv) an alcohol solvent selected from the group consisting of phenyl ethanol, ethanol, benzyl alcohol and combinations thereof, in an amount between about 0.01 and 1 percent weight/weight.

7. The composition of claim 1, wherein the composition comprises
(i) benzoic acid in an amount between about 2 and 20 percent weight/weight;
(ii) a botanical extract selected from the group consisting of portulaca extract, tetrahydocurcumanoids (THC) and derivatives thereof, and combinations thereof, in an amount between about 5 and 50 percent weight/weight;
(iii) thyme oil, thymol, or combinations thereof, in an amount between about 1 and 10 percent weight/weight; and
(iv) an alcohol solvent selected from the group consisting of phenyl ethanol, ethanol, benzyl alcohol and combinations thereof, in an amount between about 30 and 80 percent weight/weight.

8. The composition of claim 1, wherein the composition comprises
(i) benzoic acid in an amount between about 0.0001 and 0.2 percent weight/weight;
(ii) a botanical extract selected from the group consisting of Biosecur®, citrus extract, portulaca extract, tetrahydocurcumanoids (THC) and derivatives thereof, echinacea extract, and combinations thereof, in an amount between about 0.005 and 1 percent weight/weight;
(iii) an essential oil, or active constituent thereof, selected from the group consisting cinnamon oil, lemon oil, thyme oil, galangal oil, thymol, cinnamaldehyde and combinations thereof, in an amount between about 0.0001 and 1 percent weight/weight; and
(iv) an alcohol solvent selected from the group consisting of phenyl ethanol, ethanol, benzyl alcohol and combinations thereof, in an amount between about 0.01 and 1 percent weight/weight.

9. The composition of claim 1, wherein the composition comprises
(i) benzoic acid in an amount between about 0.01 and 20 percent weight/weight;
(ii) a botanical extract selected from the group consisting of Biosecur®, citrus extract, portulaca extract, tetrahydocurcumanoids (THC) and derivatives thereof, echinacea extract, and combinations thereof, in an amount between about 5 and 70 percent weight/weight;
(iii) an essential oil, or active constituent thereof, selected from the group consisting cinnamon oil, lemon oil, thyme oil, galangal oil, thymol, cinnamaldehyde and combinations thereof, in an amount between about 0.01 and 10 percent weight/weight; and
(iv) an alcohol solvent selected from the group consisting of phenyl ethanol, ethanol, benzyl alcohol and combinations thereof, in an amount between about 5 and 80 percent weight/weight.

10. The composition of claim 1, further comprising an odor masking agent.

11. The composition of claim 10, where the odor masking agent is in an amount between about 0.01 and 1 percent weight/weight.

12. The composition of claim 1, which does not further comprise lauroyl arginate, ethyl lauroyl arginate, glycerol monolaurate, glyceryl laurate or propylene glycol monolaurate.

13. The composition of claim 1, which does not further comprise antimicrobial effective amounts of any of the following antimicrobial agents: quaternary ammonium compounds, biguanides, chlorinated phenols, triclosan, chlorhexidine or chlorhexidine salt, phenoxyethanol, povidone iodine, aliphatic alcohol or parachlorometaxylenol.

14. The composition of claim 1, wherein the composition is comprised in a personal care formulation.

15. The composition of claim 14, wherein the formulation is an anionic formulation.

16. The composition of claim 14, wherein the formulation is a cationic formulation.

17. The composition of claim 1, wherein the composition is comprised in a preservative for food and beverage products, a food disinfectant, a topical disinfectant, a surface disinfectant, or an oral formulation.

18. A method of inhibiting the growth of a microbe in a personal care formulation, comprising incorporating, in said formulation, a composition comprising
(i) benzoic acid in an amount between about 0.0001 and 20 percent weight/weight;
(ii) a botanical extract selected from the group consisting of Biosecur®, citrus extract, portulaca extract, tetrahydocurcumanoids (THC) and derivatives thereof, echinacea extract, and combinations thereof, in an amount between about 0.005 and 70 percent weight/weight;
(iii) an essential oil, or active constituent thereof, selected from the group consisting cinnamon oil, lemon oil, thyme oil, galangal oil, thymol, cinnamaldehyde and combinations thereof, in an amount between about 0.0001 and 10 percent weight/weight; and
(iv) an alcohol solvent selected from the group consisting of phenyl ethanol, ethanol, benzyl alcohol and combinations thereof, in an amount between about 0.01 and 80 percent weight/weight.

19. The method of claim 18, where the microbe is selected from the group consisting of a bacterium, a yeast, a protozoan and a fungus.

20. The method of claim 18, where the microbe is a bacterium.

21. The method of claim 20, where the bacterium is selected from the group consisting of Staphylococcus aureus, Staphylococcus epidermidis, Enterococcus, Escherichia coli, and Pseudomonas aeruginosa.

22. The method of claim 18, where the microbe is Candida albicans.

23. The method of claim 18, where the microbe is Aspergillus niger.

24. A composition comprising an effective antimicrobial amount of
(i) between about 0.1-1.0% weight/weight (w/w) citrus extract selected from the group consisting of Biosecur®, grape fruit seed extract, lemon extract, orange extract and combinations thereof;
(ii) between 0.05-1.0% w/w organic acid selected from the group consisting of benzoic acid, lactic acid, citric acid and combinations thereof;
(iii) between 0.05-0.1% w/w essential oil or constituents thereof selected from the group consisting of cinnamon oil, pine oil, thymol, thyme oil, lemongrass oil, lemon oil, basil oil, oregano oil, rosemary oil, farnesol, cedarwood oil, vetiver oil. patchouli oil and combinations thereof; and
(iv) between 0.05-1.0% w/w solvent selected from the group consisting of phenyl ethanol, ethanol and combinations thereof.

25. The composition of claim 24, wherein the composition is formulated as a soap or a surface disinfectant.

26. A composition comprising an effective antimicrobial amount of
(i) benzoic acid in an amount between about 2 and 20 percent weight/weight;
(ii) a botanical extract in an amount between about 2 and 20 percent weight/weight selected from the group consisting of curcumin, tetrahydrocurcuminoid, citrus extract, Biosecur®, citrus bioflavonoids, lemon extract, and combinations thereof;
(iii) an essential oil or constituent thereof in an amount between about 1 and 20 percent weight/weight selected from the group consisting of thyme oil, thymol, carvacrol, cinnamon oil, and combinations thereof;
(iv) an alcohol solvent in an amount between about 5 and 85 percent weight/weight selected from the group consisting of phenyl ethanol, ethanol, benzyl alcohol and combinations thereof; and
(v) an odor masking agent in an amount between about 0.01 and 20 percent weight/weight selected from the group consisting of vanilla oil, vanilla oleo resin, resveratrol, safflower oil, grape seed oil, pomegranate extract, and combinations thereof.

27. The composition of claim 26, wherein the composition is comprised in a personal care product, food product or beverage product in an amount of between about 0.2-2.0% weight/weight.

28. The composition of claim 26, wherein the composition is comprised in a personal care formulation or preservative for food and beverage products.

29. A method of inhibiting the growth of a microbe in a personal care formulation, food product or beverage product comprising incorporating, in said formulation or product, a composition comprising
(i) benzoic acid in an amount between about 2 and 20 percent weight/weight;
(ii) a botanical extract in an amount between about 2 and 20 percent weight/weight selected from the group consisting of curcumin, tetrahydrocurcuminoid, citrus extract, Biosecur®, citrus bioflavonoids, lemon extract, and combinations thereof;
(iii) an essential oil or constituent thereof in an amount between about 1 and 20 percent weight/weight selected from the group consisting of thyme oil, thymol, carvacrol, cinnamon oil, and combinations thereof;
(iv) an alcohol solvent in an amount between about 5 and 85 percent weight/weight selected from the group consisting of phenyl ethanol, ethanol, benzyl alcohol and combinations thereof; and
(v) an odor masking agent in an amount between about 0.01 and 20 percent weight/weight selected from the group consisting of vanilla oil, vanilla oleo resin, resveratrol, safflower oil, grape seed oil, pomegranate extract, and combinations thereof.

30. The method of claim 29, where the microbe is selected from the group consisting of a bacterium, a yeast, a protozoan and a fungus.

31. The method of claim 29, where the microbe is a bacterium.

32. The method of claim 31, where the bacterium is selected from the group consisting of Staphylococcus aureus, Staphylococcus epidermidis, Enterococcus, Escherichia coli, and Pseudomonas aeruginosa.

33. The method of claim 29, where the microbe is Candida albicans.

34. The method of claim 29, where the microbe is Aspergillus niger.
